(19) United States
(12) Patent Application Publication
(10) Pub. No.: US 2025/0104417 A1
(43) Pub. Date: Mar. 27, 2025

(54) SYSTEMS AND METHODS FOR PROTOCOL PARAMETER DETERMINATION OF CARDIAC MAGNETIC RESONANCE IMAGING

(71) Applicant: SHANGHAI UNITED IMAGING HEALTHCARE CO., LTD., Shanghai (CN)

(72) Inventors: Wanzhou YIN, Shanghai (CN); Shiyu ZHANG, Shanghai (CN); Junpu HU, Shanghai (CN); Dong WANG, Shanghai (CN)

(73) Assignee: SHANGHAI UNITED IMAGING HEALTHCARE CO., LTD., Shanghai (CN)

(21) Appl. No.: 18/885,547

(22) Filed: Sep. 13, 2024

(30) Foreign Application Priority Data
Sep. 25, 2023 (CN) 202311245030.4

(51) Int. Cl. G06V 10/98; A61B 5/00; A61B 5/055; G01R 33/56; G06V 10/77; G06V 10/94

(52) U.S. Cl. CPC G06V 10/993; A61B 5/0037; A61B 5/0044; A61B 5/055; G01R 33/5608; G06V 10/77; G06V 10/945; G06V 2201/031

(57) ABSTRACT

Embodiments of the present disclosure provide a system for determining a protocol parameter of cardiac magnetic resonance imaging. The system includes at least one storage device storing a set of instructions and at least one processor configured to communicate with the at least one storage device. When executing the set of instructions, the at least one processor is configured to direct the system to perform operations including in response to detecting that a user operation satisfies a first condition, obtaining an inversion time scout (TI-Scout) sequence for cardiac scanning; obtaining a plurality of first scan images by performing a first scan on a target object based on the TI-Scout sequence protocol; and generating a first target parameter for a phase sensitive inversion recovery (PSIR) protocol using a target parameter determination model based on the plurality of first scan images.

### CROSS-REFERENCE TO RELATED APPLICATIONS

This application claims priority to Chinese Patent Application No. 202311245030.4, filed on Sep. 25, 2023, the entire contents of which are hereby incorporated by reference.

### TECHNICAL FIELD

The present disclosure relates to the field of medical imaging, and in particular, to systems, methods, and storage media for determining protocol parameters.

### BACKGROUND

Magnetic Resonance Imaging (MRI) is a medical imaging technology capable of generating anatomic images of human body by utilizing strong magnetic fields and radiofrequency waves. MRI has extensive applications in diagnostics and research, particularly providing rich contrast in soft tissues. During the MRI process, various imaging parameters directly influence the quality, contrast, resolution, and scan time of the generated images. Specifically, when setting up scan protocols, it is necessary for operators to have a comprehensive understanding of the principles of MRI and possess a certain level of proficiency in setting and adjusting various parameters to acquire data sequences with optimal parameters. This ensures that the imaging quality meets the requisite diagnostic and research standards.

Therefore, it is desirable to provide a system, method, and storage medium for determining a protocol parameter to obtain high-quality magnetic resonance images.

### SUMMARY

One or more embodiments of the present disclosure provides a system for determining a protocol parameter of cardiac magnetic resonance imaging. The system comprises at least one storage device storing a set of instructions and at least one processor configured to communicate with the at least one storage device. When executing the set of instructions, the at least one processor may be configured to direct the system to perform operations including in response to detecting that a user operation satisfies a first condition, obtaining an inversion time scout (TI-Scout) sequence for cardiac scanning; obtaining a plurality of first scan images by performing a first scan on a target object based on the TI-Scout sequence protocol; and generating a first target parameter for a phase sensitive inversion recovery (PSIR) protocol using a target parameter determination model based on the plurality of first scan images, the first target parameter including an inversion time (TI) corresponding to an image of myocardial inversion recovery zero-crossing, and the target parameter determination model being a machine learning model.

In some embodiments, the target parameter determination model may include an image pre-processing layer and a first image evaluation layer. Generating a first target parameter for a PSIR protocol using a target parameter determination model based on the plurality of first scan images may include generating a candidate image sequence based on the plurality of first scan images through the image pre-processing layer; and determining the first target parameter based on the candidate image sequence and a preset evaluation metric through the first image evaluation layer.

In some embodiments, the target parameter determination model may include a feature extraction layer and a second image evaluation layer. Generating a first target parameter for a PSIR protocol using a target parameter determination model based on the plurality of first scan images may include determining a first preset feature based on the plurality of first scan images; determining a candidate image feature sequence based on the first preset feature and the plurality of first scan images through the feature extraction layer; and determining the first target parameter based on the candidate image feature sequence through the second image evaluation layer.

In some embodiments, an input of the target parameter determination model may further include historically and manually selected protocol parameters.

In some embodiments, the first condition may include detecting a user's operation of selecting a first binding group. The first binding group may reflect a binding relationship between at least one parameter of the TI-Scout sequence protocol and the first target parameter.

In some embodiments, the operations may further include in response to detecting that the user's operation satisfies a second condition, obtaining a velocity encoding with scout (VENC-Scout) sequence protocol for cardiac scanning; obtaining a plurality of second scan images by performing a second scan on the target object based on the VENC-Scout sequence protocol; and generating a second target parameter for a flow quantification (FQ) protocol using the target parameter determination model based on the plurality of second scan images, the second target parameter including VENC without curling artifacts.

In some embodiments, the operations may further include in response to detecting that the user's operation satisfies a third condition, obtaining a cardiac cine MRI sequence protocol for cardiac scanning; obtaining a plurality of third scan images by performing a third scan on the target object based on the cardiac cine MRI sequence protocol; and generating a third target parameter for a coronary imaging sequence protocol using the target parameter determination model based on the plurality of third scan images, the third target parameter including a trigger delay time of a coronary phase.

In some embodiments, the operations may further include in response to detecting that the user's operation satisfies a fourth condition, obtaining a bolus tracking sequence protocol for vascular scanning; obtaining a plurality of fourth scan images by performing a fourth scan on the target object based on the bolus tracking sequence protocol; and generating a fourth target parameter for a vascular sequence protocol using the target parameter determination model based on the plurality of fourth scan images, the fourth target parameter including an instruction to automatically send a confirmation for subsequent scanning when an intensity of a vascular target region in the plurality of fourth scan images reaches an expected level.

In some embodiments, the system may further comprise a display for displaying the first target parameter marked with a binding identifier or a linking identifier, the binding identifier or the linking identifier being configured to indicate the first target parameter is shared with at least another sequence protocol.

One or more embodiments of the present disclosure provide a method for determining a protocol parameter. The method may include in response to detecting that a user operation satisfies a first condition, obtaining an inversion time scout (TI-Scout) sequence for cardiac scanning; obtaining a plurality of first scan images by performing a first scan on a target object based on the TI-Scout sequence protocol; and generating a first target parameter for a phase sensitive inversion recovery (PSIR) protocol using a target parameter determination model based on the plurality of first scan images, the first target parameter including an inversion time (TI) corresponding to an image of myocardial inversion recovery zero-crossing, and the target parameter determination model being a machine learning model.

In some embodiments, the target parameter determination model may include an image pre-processing layer and a first image evaluation layer. Generating a first target parameter for a PSIR protocol using a target parameter determination model based on the plurality of first scan images may include generating a candidate image sequence based on the plurality of first scan images through the image pre-processing layer; and determining the first target parameter based on the candidate image sequence and a preset evaluation metric through the first image evaluation layer.

In some embodiments, the target parameter determination model may include a feature extraction layer and a second image evaluation layer. Generating a first target parameter for a PSIR protocol using a target parameter determination model based on the plurality of first scan images may include determining a first preset feature based on the plurality of first scan images; determining a candidate image feature sequence based on the first preset feature and the plurality of first scan images through the feature extraction layer; and determining the first target parameter based on the candidate image feature sequence through the second image evaluation layer.

In some embodiments, an input of the target parameter determination model may further include historically and manually selected protocol parameters.

In some embodiments, the first condition may include detecting a user's operation of selecting a first binding group, the first binding group reflecting a binding relationship between at least one parameter of the TI-Scout sequence protocol and the first target parameter.

In some embodiments, the operations may further include in response to detecting that the user's operation satisfies a second condition, obtaining a velocity encoding with scout (VENC-Scout) sequence protocol for cardiac scanning; obtaining a plurality of second scan images by performing a second scan on the target object based on the VENC-Scout sequence protocol; and generating a second target parameter for a flow quantification (FQ) protocol using the target parameter determination model based on the plurality of second scan images, the second target parameter including VENC without curling artifacts.

In some embodiments, the operations may further include in response to detecting that the user's operation satisfies a third condition, obtaining a cardiac cine MRI sequence protocol for cardiac scanning; obtaining a plurality of third scan images by performing a third scan on the target object based on the cardiac cine MRI sequence protocol; and generating a third target parameter for a coronary imaging sequence protocol using the target parameter determination model based on the plurality of third scan images, the third target parameter including a trigger delay time of a coronary phase.

In some embodiments, the operations may further include in response to detecting that the user's operation satisfies a fourth condition, obtaining a bolus tracking sequence protocol for vascular scanning; obtaining a plurality of fourth scan images by performing a fourth scan on the target object based on the bolus tracking sequence protocol; and generating a fourth target parameter for a vascular sequence protocol using the target parameter determination model based on the plurality of fourth scan images, the fourth target parameter including an instruction to automatically send a confirmation for subsequent scanning when an intensity of a vascular target region in the plurality of fourth scan images reaches an expected level.

One or more embodiments of the present disclosure provide a method for cardiac scan during magnetic resonance imaging. The method may include obtaining a pre-scan protocol; obtaining a plurality of pre-scan images by performing a scan on the heart of the object based on the pre-scan protocol, the plurality of pre-scan images corresponding to a series of parameter values of the pre-scan protocol; determining a target parameter of a target protocol by performing feature recognition on the plurality of pre-scan images using a deep learning-based network; and performing magnetic resonance scanning on the heart of the object according to the target protocol. A type of the pre-scan protocol is different from that of the target protocol.

In some embodiments, determining a target parameter of a target protocol by performing feature recognition on the plurality of pre-scan images using a deep learning-based network may include obtaining a plurality of candidate image features by performing feature recognition on each of the plurality of pre-scan images using a feature recognition algorithm; determining a target image feature by evaluating the plurality of candidate image features; and determining the target parameter based on a protocol parameter of a target pre-scan image corresponding to the target image feature.

In some embodiments, determining the target parameter based on a protocol parameter of a target pre-scan image corresponding to the target image feature may include determining a quality factor of the target pre-scan image corresponding to a target image feature; in response to determining that the quality factor meets a set requirement, determining the protocol parameter of the target pre-scan image as the target parameter; and in response to determining that the quality factor does not meet the set requirement, adjusting the protocol parameter of the target pre-scan image and determining the adjusted protocol parameter as the target parameter.

### BRIEF DESCRIPTION OF THE DRAWINGS

The present disclosure is further illustrated in terms of exemplary embodiments. These exemplary embodiments are described in detail with reference to according to the drawings. These embodiments are non-limiting exemplary embodiments, in which like reference numerals represent similar structures, and wherein:

FIG. 1 is a schematic diagram illustrating an application scenario of a system for determining a protocol parameter of cardiac magnetic resonance imaging according to some embodiments of the present disclosure;

FIG. 2 is a flowchart illustrating an exemplary process for determining a protocol parameter according to some embodiments of the present disclosure;

FIG. 3 is a flowchart illustrating an exemplary process of determining a first target parameter according to some embodiments of the present disclosure;

FIG. 4 is a flowchart illustrating an exemplary process for determining at least one protocol parameter according to some embodiments of the present disclosure;

FIG. 5 is a flowchart illustrating an exemplary process for determining at least one protocol parameter according to some other embodiments of the present disclosure;

FIG. 6 is a flowchart illustrating an exemplary process for determining a protocol parameter according to some other embodiments of the present disclosure;

FIG. 7 is a flowchart illustrating an exemplary process for determining a protocol parameter according to some embodiments of the present disclosure;

FIG. 8 is a flowchart illustrating an exemplary process for determining a target protocol parameter according to some embodiments of the present disclosure;

FIG. 9 is a flowchart illustrating an exemplary process for determining a target parameter of a target protocol according to some embodiments of the present disclosure;

FIG. 10 is a flowchart illustrating an exemplary process for determining a protocol parameter according to some embodiments of the present disclosure;

FIG. 11 is a schematic diagram illustrating a device for determining a protocol parameter according to some embodiments of the present disclosure; and

FIGS. 12A-12B are schematic diagrams illustrating exemplary target image features according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system”, “device”, “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise”, “comprises”, “comprising”, “include”, “includes”, and/or “including”, merely prompt to include operations and elements that have been clearly identified, and these operations and elements do not constitute an exclusive listing. The methods or devices may also include other operations or elements.

The flowcharts used in the present disclosure illustrate operations that systems implement according to some embodiments of the present disclosure. It should be understood that the previous or subsequent operations may not be accurately implemented in order. Instead, each step may be processed in reverse order or simultaneously. Meanwhile, other operations may also be added to these processes, or a certain step or several steps may be removed from these processes.

The basic working principle of magnetic resonance imaging (MRI) is based on the phenomenon of nuclear magnetic resonance, in which the nuclei of hydrogen atoms in the body are aligned in a certain direction under a strong magnetic field. When emitted and interferes with these hydrogen nuclei, the radio frequency (RF) pulse absorbs energy and changes the alignment direction. When the RF pulse stops, these nuclei release energy and return to their original alignment, and receiving coils detect the released signals, and a computer generates images.

During MRI, image quality, contrast, resolution, and scan time are directly affected by different imaging parameters. The setting and optimization of these parameters are critical to the quality of MRI imaging. Different imaging protocols and application scenarios require targeted adjustment of these parameters for optimal imaging results and diagnostic information. For example, in brain imaging, T1 and T2 weighted imaging may have different TR and TE settings to highlight different tissue contrasts. As another example, the setting of MRI scan protocol parameters, which requires not only a deep understanding of the imaging technique and physiology but also taking into account the needs of the specific clinical application and equipment characteristics. The complexity and interactivity of these factors make the optimization of MRI imaging a complex and challenging task. Operators need to be objective in the adjustment and setup operations of the protocols. However, it may be hard for human operations to avoid subjectivity and errors, which introduces bias or even error into raw data acquired in imaging and leads to degradation of image quality or scanning failure.

In view of the foregoing, some embodiments of the present disclosure provide a method and a system for determining a protocol parameter of cardiac magnetic resonance imaging. For a specific magnetic resonance scanning scenario, a corresponding recognition algorithm is applied to a magnetic resonance scanning workflow, which may make magnetic resonance scanning more intelligent and automated while reducing the subjectivity of the setting of the protocol parameter, simplifying the magnetic resonance scanning process, and improving the image quality of imaging.

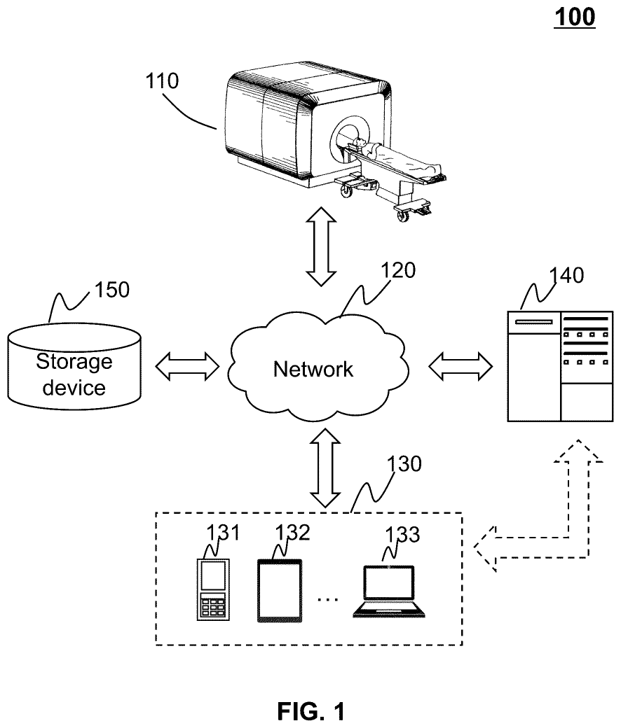

FIG. 1 is a schematic diagram illustrating an application scenario of a system for determining a protocol parameter of cardiac magnetic resonance imaging according to some embodiments of the present disclosure.

As shown in FIG. 1, the system 100 for determining the protocol parameter may include a magnetic resonance imaging (MRI) scanner 110, a processing device 120, a storage device 130, one or more terminals 140, and a network 150. In some embodiments, the MRI scanner 110, the processing device 120, the storage device 130, and/or the terminal(s) 140 may be connected and/or communicate with each other through wireless connections, wired connections, or a combination thereof. Connections between components in the system 100 may be variable. For example, the MRI scanner 110 may be connected to the processing device 120 via the network 150. As another example, the MRI scanner 110 may be directly connected to the processing device 120.

In some embodiments, the MRI scanner 110 may include a single modality imaging system and/or a multi-modality imaging system. The single modality imaging system may include the system for determining the protocol parameter. The multi-modality imaging system may include an X-ray imaging-magnetic resonance imaging (X-ray-MRI) system, a single photon emission computed tomography-magnetic resonance imaging (SPECT-MRI) system, a digital subtraction angiography-magnetic resonance imaging (DSA-MRI) system, a computed tomography magnetic resonance imaging (MRI-CT) system, a positron emission tomography-magnetic resonance Imaging (PET-MRI) system, etc.

The MRI scanner 110 may be configured to scan a target to obtain image data of the target, e.g., the MRI scanner 110 may load a sequence protocol corresponding to the target and perform scanning on the scanned target. In some embodiments, the MRI scanner 110 may include a main magnet, a gradient coil (also referred to as a spatially encoded coil), a radio frequency (RF) coil, or the like. The target scanned by the MRI scanner 110 may be biological or non-biological. For example, the target may include a patient, an artificial object, or the like. As another example, the target may include a specific portion, organ, tissue, and/or physical point of the patient. Merely by way of example, the target may include the head, brain, neck, body, shoulder, arm, chest, heart, stomach, blood vessel, soft tissue, knee, foot, or the like, or a combination thereof.

The processing device 120 may be configured to process data and/or information obtained from the MRI scanner 110, the storage device 130, and/or the terminal(s) 140. For example, the processing device 120 may obtain, in response to detecting that a user operation satisfies a first condition, an inversion time scout (TI-Scout) sequence for cardiac scanning. The processing device 120 may obtain a plurality of first scan images by controlling the MRI scanner 110 to perform a first scan on a target object based on the TI-Scout sequence. The processing device 120 may generate a first target parameter for a phase sensitive inversion recovery (PSIR) protocol using a target parameter determination model based on the plurality of first scan images. Further, the processing device 120 may control the MRI scanner 110 to image the target object based on the first target parameter. In some embodiments, the processing device 120 may be a single server or group of servers. The group of servers may be centralized or decentralized. In some embodiments, the processing device 120 may access information and/or data from the MRI scanner 110, the storage device 130, and/or the terminal 140 via the network 150. As another example, the processing device 120 may be directly connected to the MRI scanner 110, the terminal 140, and/or the storage device 130 to access the information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof.

The storage device 130 may store data, instructions, and/or any other information. In some embodiments, the storage device 130 may store data obtained from the MRI scanner 110, the processing device 120, and/or the terminal 140. In some embodiments, the storage device 130 may store data and/or instructions that the processing device 120 may perform or be used to perform the exemplary methods described in the present disclosure. In some embodiments, the storage device 130 may include a mass storage device, a removable storage device, a random access memory, a read-only memory (ROM), or the like, or a combination thereof. In some embodiments, the storage device 130 may be implemented on the cloud platform, as described elsewhere in the present disclosure.

In some embodiments, the storage device 130 may be connected to the network 150 to communicate with one or more other components (e.g., the MRI scanner 110, the processing device 120, and/or the terminal 140) of the system 100. One or more components of the system 100 may access data or instructions stored in the storage device 130 via the network 150. In some embodiments, the storage device 130 may be part of the processing device 120 or the terminal 140.

The terminal 140 may be configured to enable interaction between the user and the system 100. For example, the terminal 140 may receive instructions from the user to cause the MRI scanner 110 to scan the target. As another example, the terminal 140 may receive a processing result (e.g., a magnetic resonance cardiac image) from the processing device 120 and display the processing result to the user. In some embodiments, the terminal 140 may be connected to and/or in communication with the MRI scanner 110, the processing device 120, and/or the storage device 130. In some embodiments, the terminal 140 may include a mobile device 140-1, a tablet computer 140-2, a laptop computer 140-3, etc., or a combination thereof. In some embodiments, the terminal 140 may be part of the processing device 120 or the MRI scanner 110.

The network 150 may include any suitable network that may facilitate the exchange of information and/or data for the system 100. In some embodiments, one or more components of the system 100 (e.g., the MRI scanner 110, the processing device 120, the storage device 130, the terminal 140, etc.) may transmit information and/or data via the network with one or more other components of the system 100. As another example, the processing device 120 may obtain user instructions from the terminal 140 via the network 150. In some embodiments, the network 150 may include one or more network access points. For example, the network 150 may include a wired and/or wireless network access point, such as a base station and/or an Internet exchange point, through which one or more components of the system 100 may be connected to the network 150 to exchange data and/or information.

The foregoing description is intended to be illustrative and is not intended to limit the scope of the present disclosure. Numerous substitutions, modifications, and variations will be apparent to those skilled in the art. Features, structures, methods, and characteristics of the exemplary embodiments described herein may be combined in various ways to obtain additional and/or alternative exemplary embodiments. In some embodiments, the system 100 may include one or more additional components and may omit one or more of the components. Additionally or alternatively, two or more components of the system 100 may be integrated into a single component. For example, the processing device 120 may be integrated into the MRI scanner 110. As another example, the components of the system 100 may be replaced by another component that may fulfill the functions of the components. In some embodiments, the storage device 130 may be a data store, including a cloud computing platform, such as a public cloud, a private cloud, a community and hybrid cloud, or the like. However, these variations and modifications do not depart from the scope of the present disclosure.

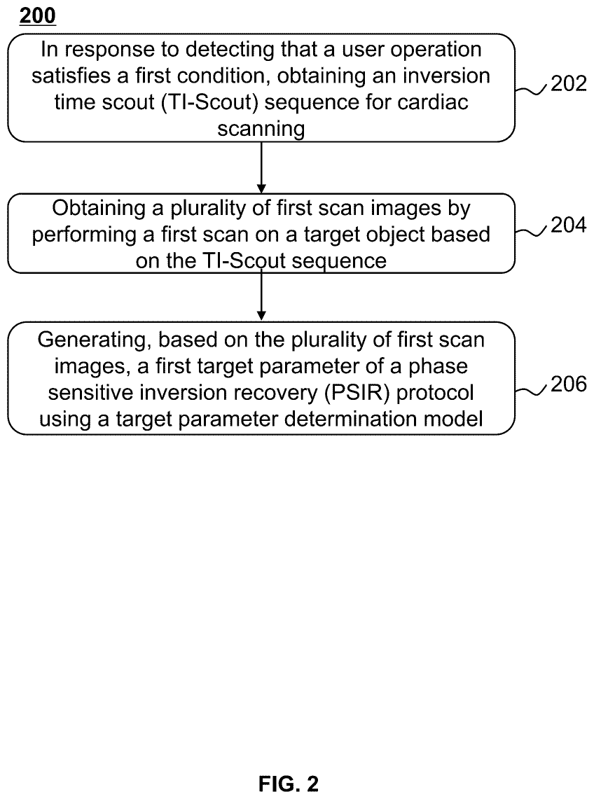

FIG. 2 is a flowchart illustrating an exemplary process for determining a protocol parameter according to some embodiments of the present disclosure. In some embodiments, the process 200 may be performed by the system 100 or a processing device (e.g., processing device 120). For example, a system for determining a protocol parameter of cardiac magnetic resonance imaging may include at least one storage device including a set of instructions and at least one processor configured to be in communication with at least one storage device. When the instructions are executed, the at least one processor is configured to instruct the system to perform a process as shown in FIG. 2. In some embodiments, process 200 may include one or more of operations described below.

In 202, in response to detecting that a user operation satisfies a first condition, an inversion time scout (TI-Scout) sequence for cardiac scanning may be obtained.

In magnetic resonance scanning, the TI-Scout sequence may be used as a pre-scan protocol to determine an optimal time point for inversion recovery of the myocardial tissue by varying an inversion time (TI). The purpose of using the TI-Scout is mainly to determine an optimal TI (e.g., a TI corresponding to an image of myocardial inversion recovery zero-crossing, which is also known as an TI corresponding to a myocardial zero point, i.e., the value of the TI at the time of passing the inversion recovery zero-crossing).

The first condition refers to a specific condition or requirement that needs to be met before obtaining the TI-Scout protocol for cardiac scanning. For example, the first condition is that the user selects a first binding group.

In some embodiments, the operation of detecting that the user operation satisfies the first condition may be detecting that the user selects the first binding group. The binding group is used to characterize the existence of parameter reuse or passing relationship between a target parameter and at least one protocol parameter of a pre-scan protocol. In some embodiments, the binding group may be characterized in the form of an operation control, a binding identifier, or a linking identifier, which is not limited by this embodiment.

In some embodiments, the first binding group reflects a binding relationship between at least one parameter of the TI-Scout sequence and a first target parameter. It may also be understood that the first binding group characterizes a reuse relationship or a passing relationship between the at least one parameter of the TI-Scout sequence and the first target parameter. The at least one parameter of the TI-Scout sequence includes the TI.

The reuse relationship or the passing relationship refers to the reuse or the determination of the at least one protocol parameter of the pre-scan protocol as the target parameter. For example, the TI corresponding to the image of myocardial inversion recovery zero-crossing of the reversal time tracking sequence protocol of the TI-Scout sequence is reused or determined as the target parameter of a target protocol. The target protocol refers to a scan protocol used to perform scanning (which may be referred to as formal scanning) after pre-scanning, and the target parameter is one of the protocol parameters of the target protocol.

In some embodiments, the user may select the first binding group in a plurality of ways, such as clicking, dragging, gesturing, voicing, etc. If the operation of the user selecting the first binding group is detected, the processing device may obtain the TI-Scout sequence that may be used for cardiac scanning based on an indication of the binding relationship of the first binding group.

In some embodiments, upon detecting that the user's operation satisfies the first condition, the processing device may obtain the TI-Scout sequence for cardiac scanning by loading a predetermined scan protocol.

In 204, a plurality of first scan images may be obtained by performing a first scan on a target object based on the TI-Scout sequence.

The target object is a scan target (or part of the target). The target object may be biological or non-biological. For example, the target object may include a patient, an artificial object, etc. As another example, the target object may include a specific portion, organ, tissue, and/or physical point of the patient. Merely by way of an example, the target may include the head, brain, neck, body, shoulder, arm, chest, heart, stomach, blood vessel, soft tissue, knee, foot, or the like, or a combination thereof.

The first scan is a pre-scan of the target object performed based on the TI-Scout sequence. A first scan image refers to an image obtained by the MRI scanner from the first scan of the target object after loading the TI-Scout sequence. A count of the plurality of first scan images may be one or more.

In some embodiments, after the TI-Scout sequence is loaded, the user may slightly adjust parameters (e.g., a scanning range, a resolution, etc.) according to conditions of the target object (such as the body size), after which the first scan may be initiated to obtain the plurality of first scan images.

In 206, based on the plurality of first scan images, a first target parameter of a phase sensitive inversion recovery (PSIR) protocol may be generated using a target parameter determination model.

In magnetic resonance imaging, the PSIR protocol refers to a sequence protocol that may be used to enhance the contrast and resolution of a target region and to distinguish different tissue types through phase information. For example, the target region may include the heart muscle, scars, or the like.

In some embodiments, the PSIR protocol may be predetermined. For

example, a scan protocol corresponding to the first target parameter may be determined to be the PSIR protocol when the user selects and determines the first binding group, or a target protocol corresponding to a pre-scan protocol during current scanning may be determined to be the PSIR protocol. In the same or different application scenarios, the pre-scan protocol and the target protocol may be different. For example, if the pre-scan protocol is a TI-Scout sequence, the target protocol may be a PSIR protocol. If the pre-scan protocol is a velocity encoding with scout (VENC-Scout) sequence, the target protocol may be a flow quantification (FQ) protocol. If the pre-scan protocol is a cardiac cine MRI sequence, the target protocol may be a coronary imaging sequence. Descriptions of other pre-scan protocols and target protocols may be found hereinafter.

The target parameter refers to a reusable or passable protocol parameter obtained after performing a pre-scan. A count of the target parameter may be one or more. The first target parameter refers to a protocol parameter determined based on results of the first scan performed based on the TI-Scout sequence protocol in an application scenario of cardiac scanning. For example, the first target parameter may include the TI corresponding to the image of myocardial inversion recovery zero- crossing.

The target parameter determination model refers to a tool that learns from data using training algorithms, which may be used to predict new data. For example, the target parameter determination model is a machine learning model, including a convolutional neural network model, a recurrent neural network model, or the like.

In some embodiments, the processing device may input the plurality of first scan images to the target parameter determination model, and the target parameter determination model may process the plurality of first scan images to output the first target parameter of the TI-Scout sequence.

In some embodiments, an input of the target parameter determination model may include, in addition to the first scan images, historically and manually selected protocol parameters and/or a physiological state of a patient.

The historically and manually selected protocol parameters refer to target parameters that are manually selected by the doctor during historical scanning when the scanning is performed using the target protocol (e.g., the PSIR protocol). Adding the target parameters that are manually selected by the doctor during the historical scanning using the target protocol to the input of the target parameter determination model may provide an accurate reference to the target parameter determination model, thus improving the accuracy of model prediction.

The physiological state of the patient includes the heart rate, respiration and exercise status, etc., of the target object. When performing MRI scanning, changes in the patient's heart rate, and respiration and exercise state may affect the final imaging quality. When determining the target parameter, inputting the physiological state of the patient into the target parameter determination model gives a reference for the target parameter determination model providing the physiological state of the patient, and thus predict the target parameter more accurately.

In some embodiments, the target parameter determination model may be obtained by model training with a plurality of pieces of first training data. Each piece of first training data may include a first training sample and a first label corresponding to the first training sample. The first training sample includes a plurality of first sample scan images from historical data corresponding to historical scanning. The first label includes a first historical target parameter used in the target protocol corresponding to the historical scan from the historical data. The first historical target parameter may be obtained by manual labeling. In some embodiments, specific manners of model training may include stochastic gradient descent algorithm, mini-batch gradient descent algorithm, or the like.

In some embodiments, the target parameter determination model may include an image pre-processing layer and a first image evaluation layer. The target parameter determination model may generate a candidate image sequence based on the plurality of first scan images through the image pre-processing layer, and determine the first target parameter based on the candidate image sequence and a preset evaluation metric through the first image evaluation layer.

The image pre-processing layer refers to a network layer used to perform image pre-processing on the plurality of first scan images in the target parameter determination model. The image pre-processing may include image filtering, artifact correction, segmentation, image enhancement, or the like.

The candidate image sequence includes a plurality of images obtained by pre-processing the plurality of first scan images. In some embodiments, the plurality of first scan images may be pre-processed by the image pre-processing layer to obtain the candidate image sequence after inputting the plurality of first scan images into the target parameter determination model.

The preset evaluation metric refers to a parameter or a numerical value set to quantify the image quality to objectively evaluate the image quality. In some embodiments, the preset evaluation metric may include one or more of spatial resolution, blurriness, whether a complete target area is included, and whether a preset condition is satisfied. The preset condition may include one or more of a myocardial zero point, a curling artifacts-free feature, a trigger delay time of a coronary phase, and whether a brightness reaches a threshold. The preset evaluation metric may be set by the user according to actual needs.

The first image evaluation layer refers to a network layer connected to the image pre-processing layer in the target parameter determination model. The first image evaluation layer may be configured to predict the first target parameter based on the candidate image sequence and the preset evaluation metric. For example, the candidate image sequence and the preset evaluation metric may be input together to the first image evaluation layer, and the first image evaluation layer may output a predicted first target parameter.

In this embodiment, by performing image pre-processing on the first scan image, information such as noise that may affect the subsequent image evaluation may be removed or reduced. At the same time, the preset evaluation metric is introduced to serve as a reference for the first image evaluation layer in analyzing and processing the information, thereby improving the prediction accuracy of the first target parameter.

In some embodiments, the target parameter determination model may further include an evaluation metric selection layer. The evaluation metric selection layer may be configured to determine the preset evaluation metric based on the scan protocol and the protocol parameter contained therein, thereby reducing the influence of human subjective factors. For example, the target parameter determination model may determine the preset evaluation metric based on the TI-Scout sequence and at least one protocol parameter included therein, and the candidate image sequence, through the evaluation metric selection layer.

In some embodiments, the protocol parameter of the TI-Scout sequence may include at least one of a TI, a return time, a repetition time, a flip angle, and an acquisition matrix. In some embodiments, at least one protocol parameter included in the TI-Scout sequence may include at least the TI.

The evaluation metric selection layer is an optional structure in the target parameter determination model. For example, if the target parameter determination model does not include the evaluation metric selection layer, the preset evaluation metric may be predetermined and directly input into the target parameter determination model when used.

In some embodiments, the TI-Scout sequence and at least one protocol parameter included therein, and the candidate image sequence may be input to the evaluation metric selection layer after outputting the preset evaluation metric by the evaluation metric selection layer.

Then, the first target parameter may be determined based on the candidate image sequence and the preset evaluation metric, via the first image evaluation layer. For example, the candidate image sequence and the preset evaluation metric may be inputted to the first image evaluation layer for processing, then the first target parameter may be outputted.

In some embodiments, the target parameter determination model may further include one or more other layers, e.g., an input layer, an output layer, a feature extraction layer, a second image evaluation layer, or the like.

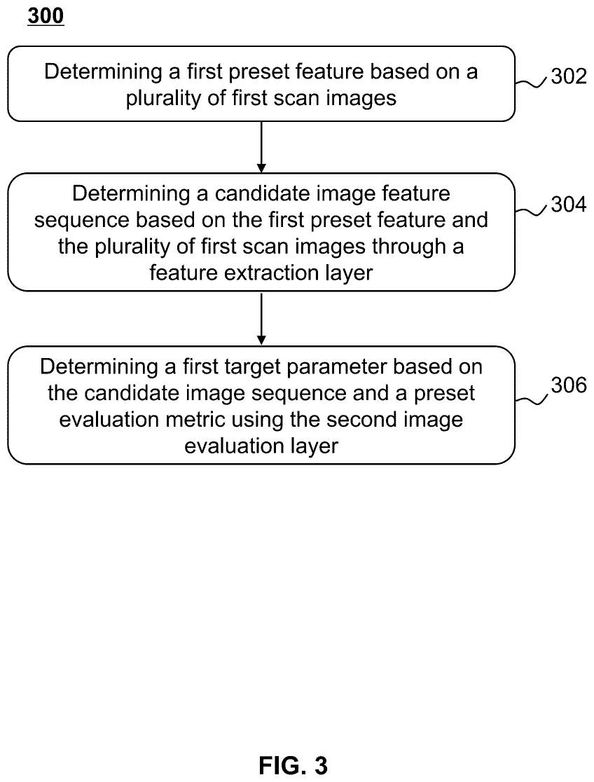

More descriptions regarding determining the first target parameter may be found in the related description of FIG. 3 hereinafter.

In some embodiments of the present disclosure, for a specific application scenario of cardiac scanning, the TI-Scout sequence is used as the pre-scan protocol to obtain the plurality of first scan images during the pre-scanning. Then, the plurality of first scan images are processed by the target parameter determination model to determine the first target parameter of the target protocol (the PSIR protocol) for the subsequent scanning, which makes the magnetic resonance scanning more intelligent and automated, and at the same time reduces the subjectivity of the setting of the target protocol parameter and the dependence on the user's experience, simplifies the magnetic resonance scanning process, and facilitates the operation.

FIG. 3 is a flowchart illustrating an exemplary process of determining a first target parameter according to some embodiments of the present disclosure. In some embodiments, process 300 may be performed by a system for determining a protocol parameter of cardiac magnetic resonance imaging or a processing device (e.g., the processing device 120). For example, the system for determining the protocol parameter may include at least one storage device including a set of instructions and at least one processor configured to be in communication with the at least one storage device. When the instructions are executed, the at least one processor is configured to instruct the system to perform operations shown in FIG. 3. In some embodiments, operation 206 may be performed according to the process 300, which may include one or more of the following operations.

In some embodiments, a target parameter determination model may include a feature extraction layer and a second image evaluation layer.

In 302, a first preset feature may be determined based on a plurality of first scan images.

The first preset feature refers to a feature that the feature extraction layer needs to extract from the first scan image. In some embodiments, the first preset feature may include one or more of a signal-to-noise ratio, a contrast ratio, a brightness, the presence of artifacts, and a uniformity of the image.

In some embodiments, the target parameter determination model may further include a feature selection layer. The feature selection layer may be configured to determine the first preset feature based on the plurality of first scan images.

In some embodiments, the processing device may determine the first preset feature based on the TI-Scout sequence and at least one protocol parameter included therein, and the plurality of first scan images, through the feature selection layer. For example, after inputting the TI-Scout sequence and the at least one protocol parameter included therein, and the plurality of first scan images to the feature selection layer, the first preset feature may be outputted by the feature selection layer.

The first preset feature may be used to indicate which image features are to be extracted by the feature extraction layer from the plurality of first scan images.

In 304, a candidate image feature sequence may be determined through the feature extraction layer based on the first preset feature and the plurality of first scan images.

The candidate image feature sequence refers to an image feature sequence extracted from the first scan image. In some embodiments, the candidate image feature sequence may be represented as a feature vector.

In some embodiments, the processing device may input the first preset feature and the plurality of first scan images to the feature extraction layer. The feature extraction layer may extract, based on an indication of the first preset feature, from the plurality of first scan images, the candidate image feature sequence.

In some embodiments, the feature extraction layer may include a plurality of feature extraction sub-layers. The processing device may determine a target feature extraction sub-layer from the plurality of feature extraction sub-layers based on the TI-Scout sequence and at least one protocol parameter included therein. The processing device may determine the candidate image feature sequence based on the first preset feature and the plurality of first scan images through the target feature extraction sub-layer.

The target feature extraction sub-layer refers to the one selected from the plurality of feature extraction sub-layers that is most compatible with the target protocol, which may also be understood as the one with the highest matching degree. For example, for different pre-scan protocols, features to be extracted from the pre-scan image in determining the target parameter may be different. The feature selection layer may be configured to determine which features need to be extracted from the image based on the type of the pre-scan protocol and to represent the need in the form of the first preset feature (e.g., in the form of a vector). The feature extraction layer performs feature extraction from the pre-scan image based on the features selected by the feature selection layer.

Exemplarily, the currently adopted pre-scan protocol is the TI-Scout sequence, and features of the first scan images obtained by scanning based on the pre-scan protocol include a signal-to-noise ratio, a contrast ratio, a brightness, the presence of artifacts, uniformity, myocardial features, the presence of convolutions, coronary motion features, or the like. However, the target protocol may not need all of the features, and it is possible to select, through the feature selection layer, the features that need to be extracted and instruct the feature extraction layer to extract the features from the first scan image.

In this way, indicating the image features to be extracted by the first preset feature may enable the extracted features to be more targeted, reduce the number of irrelevant features, and thus improve the accuracy of the model prediction.

In 306, the first target parameter may be determined based on the candidate image sequence and the preset evaluation metric using the first image evaluation layer.

In some embodiments, the processing device may input the candidate image feature sequence to a second image evaluation layer, which outputs the first target parameter.

It should be noted that in the target parameter determination model, the first image evaluation layer and the second image evaluation layer described above may be the same network layer or different network layers. If the first image evaluation layer and the second image evaluation layer are different network layers, the target parameter determination model may weigh the target parameter output from the first image evaluation layer and the second image evaluation layer, thereby obtaining a final first target parameter. Weights of the weighting may be determined according to the actual need, which is not limited in the present embodiment.

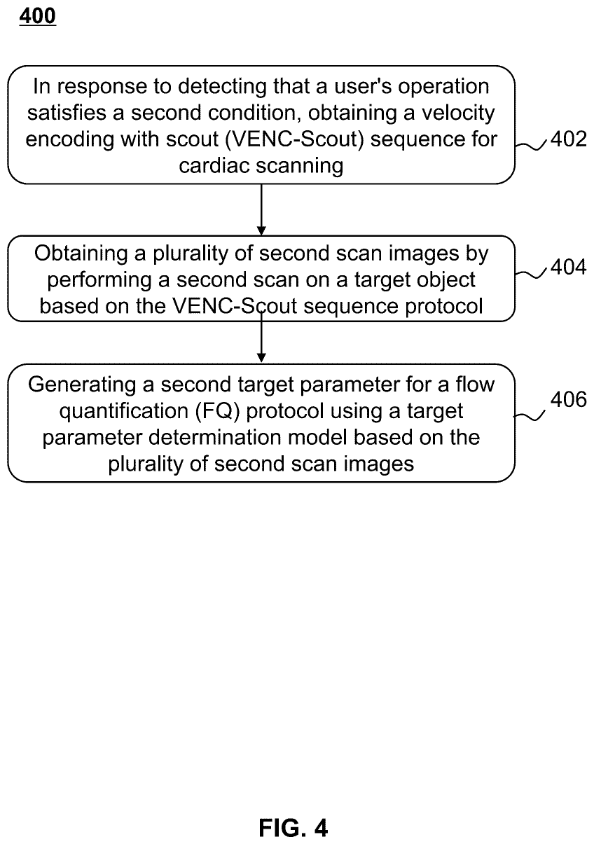

FIG. 4 is a flowchart illustrating an exemplary process for determining at least one protocol parameter according to some embodiments of the present disclosure. In some embodiments, process 400 may be performed by a system for determining a protocol parameter of cardiac magnetic resonance imaging or a processing device (e.g., the processing device 120). For example, a system for determining a protocol parameter of cardiac magnetic resonance imaging may include at least one storage device including a set of instructions and at least one processor configured to be in communication with at least one storage device. When the instructions are executed, the at least one processor is configured to instruct the system to perform operations shown in FIG. 4. In some embodiments, the process 400 may include one or more of the operations described below.

In 402, in response to detecting that a user's operation satisfies a second condition, a velocity encoding with scout (VENC-Scout) sequence protocol for cardiac scanning may be obtained.

The VENC-Scout sequence protocol refers to a technique for assessing a blood flow velocity in magnetic resonance imaging for cardiac scanning, which may detect and estimate a velocity of a substance in flow (e.g., blood) by determining a velocity-encoded parameter.

The second condition refers to a specific condition or requirement that needs to be fulfilled before obtaining the VENC-Scout sequence protocol for cardiac scanning. For example, the second condition is that the user selects a second binding group. In some embodiments, the second condition includes detecting an action of the user selecting the second binding group.

In some embodiments, the second binding group reflects a binding relationship between at least one parameter of the VENC-Scout sequence protocol and a second target parameter. For example, the second binding group reflects a reuse relationship or a passing relationship between the at least one parameter of the VENC-Scout sequence protocol and the second target parameter. The at least one parameter of the VENC-Scout sequence protocol includes a VENC value.

In some embodiments, the target protocol bound to the VENC-Scout sequence protocol and having the reuse relationship or the passing relationship with VENC-Scout sequence protocol is a flow quantification (FQ) protocol. The FQ protocol refers to an MRI protocol for the quantitative measurement of velocity and flow parameters of substances in flow (e.g., blood). The second target parameter for reuse or passing is a VENC without curling artifacts, which is the most appropriate VENC parameter corresponding to a pre-scanned image without curling artifacts.

In 404, a plurality of second scan images may be obtained by performing a second scan on a target object based on the VENC-Scout sequence protocol. More descriptions regarding operation 404 may be found in the previous description related to operation 204, and the difference between the two is that the pre-scan sequences used are different, and the other operations performed may be similar or the same.

In 406, a second target parameter for a flow quantification (FQ) protocol may be generated using the target parameter determination model based on the plurality of second scan images. In some embodiments, the second target parameter includes the VENC without curling artifacts. The target parameter determination model is a machine learning model.

It should be noted that the target parameter determination model for determining the second target parameter and the target parameter determination model for determining the first target parameter may be the same or different models. Using the same model may effectively reduce the difficulty of model training and conserve model resources, whereas using a different model may be more targeted, and may improve the accuracy of the prediction of the target parameter when facing different target protocols.

More descriptions regarding operation 406 may be found in the preceding description related to operation 206. The difference between the two is that the target sequences to which the target parameters are directed are different. In determining the second target parameter, other operations performed may be the same as those performed in determining the first target parameter.

In some embodiments of the present disclosure, for a specific application scenario of cardiac scanning, the VENC-Scout sequence protocol is used as the pre-scan protocol to obtain the plurality of second scan images during the pre-scanning. Then, the plurality of second scan images are processed by the target parameter determination model to determine the second target parameter of the target protocol (the FQ protocol) for the subsequent scanning, which makes the magnetic resonance scanning more intelligent and automated, and at the same time reduces the subjectivity of the setting of the target protocol parameter and the dependence on the user's experience, simplifies the MRI process, and facilitates the operation.

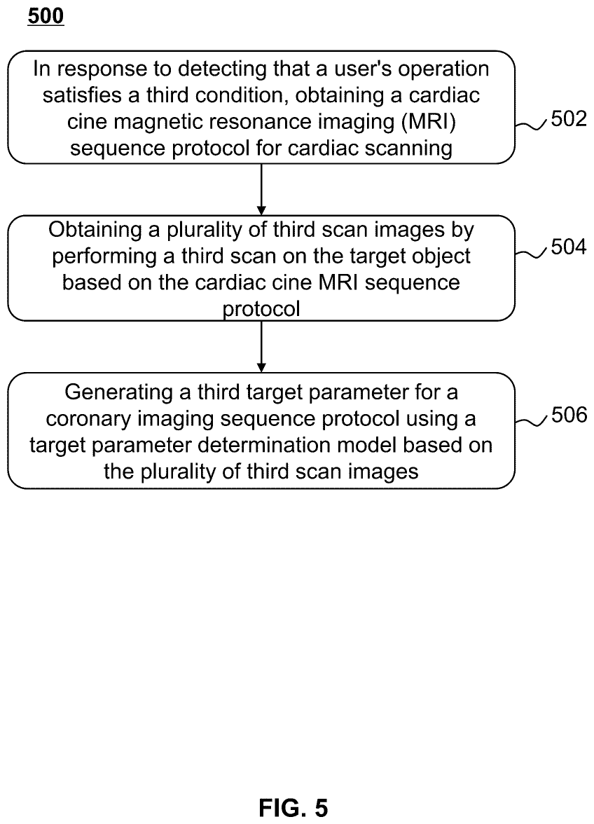

FIG. 5 is a flowchart illustrating an exemplary process for determining at least one protocol parameter according to some other embodiments of the present disclosure. In some embodiments, process 500 may be performed by a system for determining a protocol parameter of cardiac magnetic resonance imaging or a processing device (e.g., the processing device 120). For example, the system for determining the protocol parameter may include at least one storage device including a set of instructions and at least one processor configured to be in communication with at least one storage device. When the instructions are executed, the at least one processor is configured to instruct the system to perform operations shown in FIG. 5. In some embodiments, the process 500 may include one or more of the operations described below.

In 502, in response to detecting that a user's operation satisfies a third condition, a cardiac cine magnetic resonance imaging (MRI) sequence protocol for cardiac scanning may be obtained.

The cardiac cine MRI sequence protocol refers to an MRI scanning sequence that captures the heart's activity at a plurality of time points through dynamic imaging techniques, visualizing the heart's movements.

The third condition refers to a specific condition or requirement that needs to be met before obtaining the cardiac cine MRI sequence protocol for cardiac scanning. For example, the third condition selects a third binding group for the user. In some embodiments, the third condition includes detecting an action of a user selecting the third binding group. In some embodiments, the third binding group reflects a binding relationship between at least one parameter of the cardiac cine MRI sequence protocol and a third target parameter. For example, the third binding group reflects a reuse relationship or a passing relationship between the at least one parameter of the cardiac cine MRI sequence protocol and the third target parameter. The at least one parameter of the cardiac cine MRI sequence protocol includes a trigger delay time.

In some embodiments, a target protocol that is bound to a velocity encoding with scout (VENC-Scout) sequence protocol and has the reuse relationship or the passing relationship with the VENC-Scout sequence protocol is a coronary imaging sequence protocol. The coronary imaging sequence protocol refers to a protocol for obtaining an MRI imaging sequence of detailed anatomical and functional information of coronary, which is specifically used in detecting coronary disease. The third target parameter for reuse or passing is the trigger delay time of the coronary phase or views per segment (VPS). The VPS refers to a count of K-spaces obtained during each cardiac cycle. The VPS may be obtained by calculating the trigger delay time of the coronary phase, which may be calculated outside of the target parameter determination model or within the target parameter determination model.

In 504, a plurality of third scan images may be obtained by performing a third scan on a target object based on the cardiac cine MRI sequence protocol. More descriptions regarding operation 504 may be found in the previous description related to operation 204, where the difference between the two is in the pre-scan sequences used, and the other operations performed may be the same.

In 506, a third target parameter for the coronary imaging sequence protocol is generated using the target parameter determination model based on the plurality of third scan images.

In some embodiments, the third target parameter includes the trigger delay time of the coronary phase. The target parameter determination model is a machine learning model. It is noted that the target parameter determination model for determining the third target parameter may be the same or different from the target parameter determination model for determining the first target parameter.

More descriptions regarding operation 506 may be found in the preceding description related to operation 206, where the difference between the two is that the target sequences to which the target parameters are directed are different, and in determining the third target parameter, other operations performed may be the same as those performed in determining the first target parameter.

In some embodiments of the present disclosure, for a specific application scenario of cardiac scanning, a cardiac cine MRI sequence is used as the pre-scan protocol to obtain the plurality of third scan images during the pre-scanning. Then, the plurality of third scan images are processed by the target parameter determination model to determine the third target parameter of the target protocol (the coronary imaging protocol) for the subsequent scanning, which makes the magnetic resonance scanning more intelligent and automated, and at the same time reduces the subjectivity of the setting of the target protocol parameter and the dependence on the user's experience, simplifies the magnetic resonance scanning process, and facilitates the operation.

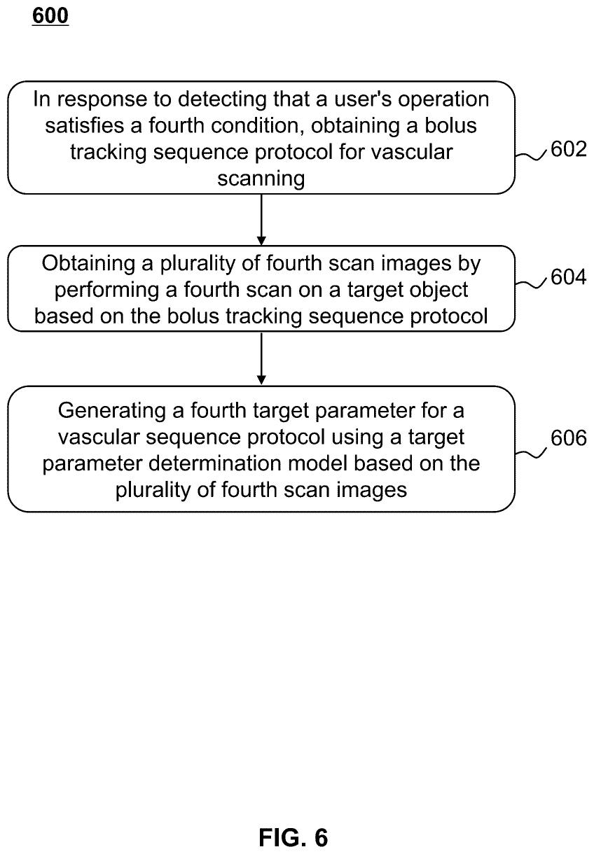

FIG. 6 is a flowchart illustrating an exemplary process for determining a protocol parameter according to some other embodiments of the present disclosure. In some embodiments, process 600 may be performed by a system for determining a protocol parameter of cardiac magnetic resonance imaging or a processing device (e.g., the processing device 120). For example, the system for determining the protocol parameter may include at least one storage device including a set of instructions and at least one processor configured to be in communication with the at least one storage device. When the instructions are executed, the at least one processor is configured to instruct the system to perform operations shown in FIG. 6. In some embodiments, the process 600 may include the operations described below.

In 602, in response to detecting that the user's operation satisfies a fourth condition, a bolus tracking sequence protocol for vascular scanning may be obtained.

A bolus tracking sequence refers to a pre-scan sequence used to monitor changes in the concentration of a contrast agent in blood vessels. In enhanced vascular scanning, by injecting the contrast agent, the contrast tracking sequence may detect in real-time changes in the concentration of the drug in the artery, usually manifested as a change in brightness on the image.

The fourth condition refers to a specific condition or requirement that needs to be fulfilled before the bolus tracking sequence protocol for vascular scanning. For example, the fourth condition selects a fourth binding group for a user.

In some embodiments, the fourth condition includes detecting an action of the user selecting the fourth binding group. In some embodiments, the fourth binding group reflects a binding relationship between at least one parameter of the bolus tracking sequence protocol and the fourth target parameter. For example, the fourth binding group reflects a reuse relationship or a passing relationship between the at least one parameter of the bolus tracking sequence protocol and the fourth target parameter. The at least one parameter of the bolus tracking sequence protocol includes a frame where a vascular region of interest (ROI) intensity reaches expectation. For example, the clarity of the vascular ROI reaches an expected level.

In some embodiments, a starting time of scanning after drug application is important in vascular enhancement scanning. Inappropriate selection of the starting time for scanning may cause the contrast of generated image(s) not to meet clinical requirements. Therefore, the bolus tracking sequence protocol may be used in scanning before the vascular enhancement scanning. The bolus tracking sequence protocol has a faster scanning speed and refreshes one frame of the image at the same time interval. The brightness of the blood vessels in the image may increase with the increase of the drug concentration. In the past, technicians may manually trigger the scanning using the vascular protocol after observing (with naked eyes) the brightness of the blood vessels reaching an empirical value. In this embodiment, the technicians only need to select one vascular ROI, changes in the vascular ROI intensity may be automatically detected using an algorithm, and the next vascular protocol scanning may be triggered. The frame where the vascular ROI intensity reaches expectation refers to an image in which the brightness of the blood vessels detected using the algorithm reaches a preset value in a plurality of frames.

In some embodiments, a target protocol bound to the bolus tracking sequence protocol and having the parameter reuse relationship or passing relationship with the bolus tracking sequence protocol is a vascular sequence protocol. The fourth target parameter for reuse or passing is an instruction to automatically send a confirmation for subsequent scanning after the ROI intensity in the pre-scan image reaches an expected level.

In 604, a plurality of fourth scan images may be obtained by performing a fourth scan on a target object based on the bolus tracking sequence protocol. More descriptions regarding operation 604 may be found in the previous description related to operation 204, where the difference between the two is in the pre-scan sequences used, and the other operations performed may be the same.

In 606, a fourth target parameter may be generated for the vascular sequence protocol using the target parameter determination model based on the plurality of fourth scan images.

In some embodiments, the fourth target parameter includes an instruction to automatically send a confirmation for subsequent scanning when an intensity of a vascular target region in the plurality of fourth scan images reaches an expected level. The target parameter determination model is a machine learning model. It is noted that the target parameter determination model for determining the fourth target parameter may be the same or a different model than the target parameter determination model for determining the first target parameter.

More descriptions regarding operation 606 may be found in the preceding description related to operation 206, where the difference is that the target sequences directed by the target parameters are different, and in determining the fourth target parameter, other operations performed may be the same as those performed in determining the first target parameter.

In some embodiments of the present disclosure, for a specific application scenario of cardiac scanning, the bolus tracking sequence protocol is used as the pre-scan protocol to obtain the plurality of fourth scan images during the pre-scanning. Then, the plurality of fourth scan images are processed by the target parameter determination model to determine the fourth target parameter of the target protocol (the vascular sequence) for the subsequent scanning, which makes the magnetic resonance scanning more intelligent and automated, and at the same time reduces the subjectivity of the setting of the target protocol parameter and the dependence on the user's experience, simplifies the magnetic resonance scanning process, and facilitates the operation.

In order to facilitate a clearer understanding of the scan sequence involved in the embodiments of the present disclosure, several types of scan protocols are explained below.

An inversion time scout (TI-Scout) sequence refers to a pre-scan protocol for MRI, which determines an optimal time point for inversion recovery of the myocardial tissue by varying an inversion time (TI). The TI-Scout sequence includes the following features. Myocardial features, which are specific features or attributes about the myocardium (the myocardial tissue), may be detected and analyzed by image recognition techniques. A myocardial zero point, which is a specific time point at which the myocardial tissue has zero signal intensity during inversion recovery. An image of myocardial inversion recovery zero-crossing, which is an image reflecting the myocardial tissue with zero signal intensity during inversion recovery. An inversion time (TI) corresponding to the image of myocardial inversion recovery zero-crossing, which corresponds to the myocardial zero point, i.e., a TI value during myocardial inversion recovery zero-crossing.

A phase sensitive inversion recovery (PSIR) protocol, which is an MRI imaging technique used to enhance contrast and resolution and to differentiate between different tissue types by phase information, especially for myocardial imaging. The PSIR protocol includes the following features. A velocity encoding with scout (VENC-Scout) sequence, which is a pre-scan protocol for MRI, detects and estimates a velocity of a substance in flow (e.g., blood) by determining a VENC parameter. Curling artifacts, which are artifacts or distortions that appear in the image and are generally caused by errors in data acquisition or processing. Without curling artifacts means that no such artifacts or distortions appear. A curling artifacts-free feature, which is a feature in which no curling artifacts or distortion appear in the image. An image without curling artifacts, which is a clear image without curling artifacts or distortion. The VENC without curling artifacts, which is the most appropriate VENC parameter obtained in the absence of the curling artifacts.

A flow quantification (FQ) protocol, which is an MRI protocol for the quantitative measurement of velocity and flow parameters of a substance in flow (e.g., blood). The FQ protocol includes the following features. A cardiac cine MRI sequence protocol, which is an MRI scan sequence that captures the heart's activity at a plurality of time points using dynamic imaging techniques to “film” the heart's movements. Coronary motion features, which is information about motion patterns and features of the coronary (the arteries responsible for blood supply to the heart), such as a trajectory of the arteries. A cine MRI image corresponding to a trigger delay time of a coronary phase, which is an image recording the coronary at various time points from the start to the end of the motion in the cardiac cine MRI sequence. The trigger delay time of the coronary phase, which is the time at which the coronary start and end during a specific time period in the cycle of cardiac motion.

A coronary imaging sequence, which is an MRI imaging sequence specifically designed to obtain detailed anatomical and functional information about the coronary, and is primarily used to detect coronary disease. The coronary imaging sequence includes the following features. A bolus tracking sequence, which is a pre-scan sequence used to monitor changes in the concentration of a contrast agent in the blood vessels. In enhanced vascular scanning, by injecting the contrast agent, the contrast tracking sequence may detect in real-time changes in the concentration of the drug in the artery, usually manifested as a change in brightness on the image. Doctors currently determine when to perform formal vascular sequence scanning by observing changes in brightness on the image of the sequence with the naked eyes.

It should be noted that the method for determining the protocol parameter disclosed in some embodiments of the present disclosure involves determining the first target parameter, the second target parameter, the third target parameter, and the fourth target parameter. In practice, any one of the target parameters (e.g., the first target parameter, the fourth target parameter, etc.) may be determined based on actual scan needs, or a plurality of target parameters may be determined therein.

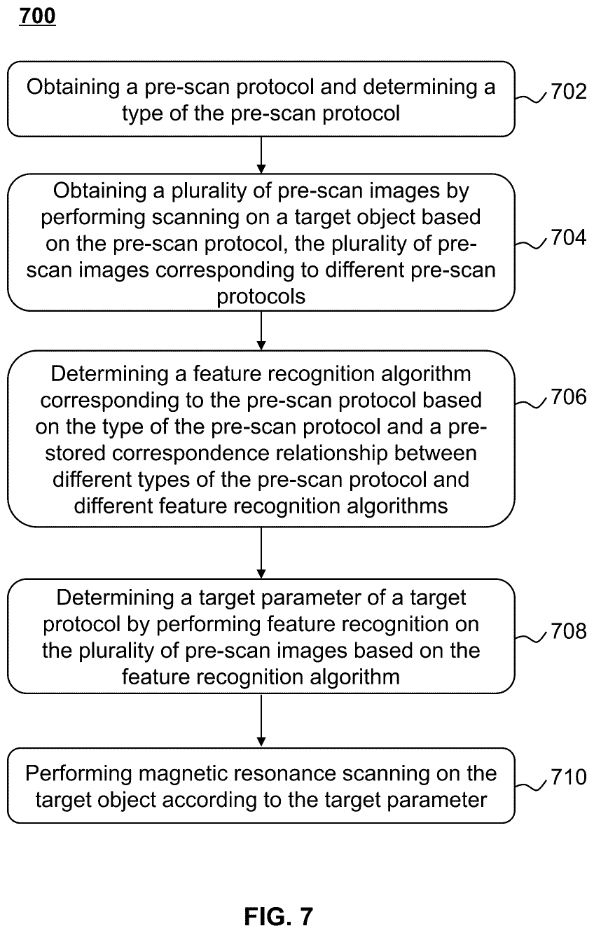

FIG. 7 is a flowchart illustrating an exemplary process for determining a protocol parameter according to some embodiments of the present disclosure. In some embodiments, process 700 may be performed by a system for determining a protocol parameter of cardiac magnetic resonance imaging or a processing device (e.g., the processing device 120). For example, a system for determining a protocol parameter of cardiac magnetic resonance imaging may include at least one storage device including a set of instructions and at least one processor configured to be in communication with the at least one storage device. When the instructions are executed, the at least one processor is configured to instruct the system to perform operations shown in FIG. 7. In some embodiments, the process 700 is a generalized illustration of the processes shown in FIGS. 2-6, and the process 700 may include one or more of the operations described below.

In 702, a pre-scan protocol may be obtained, and a type of the pre-scan protocol may be determined. In some embodiments, operation 702 may correspond to the operation of obtaining the pre-scan protocol hereinabove (e.g., operation 202, operation 402, operation 502, and operation 602).

In practice, the magnetic resonance scanning includes positioning of the target object, setting protocol parameter, and starting the scan. Positioning of the target object may refer to moving a detection bed (on which the target object is supported) in the magnetic resonance device so that a target scan part of the target object is located at an isocenter of the magnetic resonance device. The image quality ultimately obtained by the magnetic resonance scanning is closely related to each operation in the magnetic resonance scanning. In particular, in the setting of the protocol parameter, the protocol parameter needs to be set accordingly to enable a sequence to collect data with an optimal parameter, thus ensuring that the quality of the magnetic resonance imaging meets the corresponding diagnostic requirement. Accordingly, in the embodiments of the present disclosure, a user may enter a protocol parameter determination instruction into the magnetic resonance device before formally scanning the target object using the magnetic resonance device. The magnetic resonance device scans the target object based on the protocol parameter determination instruction. The user refers to an operator who performs the corresponding operation of the magnetic resonance device. The target object refers to an object that needs magnetic resonance imaging.

In an embodiment of the present disclosure, the magnetic resonance device detects the protocol parameter determination instruction, which may include the magnetic resonance device detecting a preset operation for the magnetic resonance device. The preset operation may be set according to the actual need and is not limited herein. Exemplarily, the preset operation may be clicking a preset control on the magnetic resonance device. Based on this, the magnetic resonance device, in response to detecting that the preset control is clicked, indicates that the preset operation is detected, i.e., the protocol parameter determination instruction described above is detected.

Based on this, the magnetic resonance device may obtain a pre-scan protocol after detecting the protocol parameter determination instruction. The pre-scan protocol may be one or more than one, without limitation herein. In some embodiments, the preset operation detected by the magnetic resonance device may be an operation for which the user selects a preset binding group.

It should be noted that the pre-scan protocol may include the setting of parameters including a radio frequency (RF) pulse, a gradient field, and a signal acquisition moment and an arrangement thereof in a time sequence, i.e., the pre-scan protocol determines an arrangement of variations of the RF pulse and the gradient field in the time sequence. The pre-scan protocol may include one or more of a repetition time, an echo time, an effective echo time, an echo chain length, an echo gap, an inversion time, the number of excitations, an acquisition time, or the like. For example, the pre-scan protocol may include a bandwidth, an amplitude, time to apply, and a duration of the RF pulse. The pre-scan protocol may include a time of application of the gradient field and a duration of the gradient field.

In some embodiments of the present disclosure, the magnetic resonance device may determine the type of scan protocol based on the name of the pre-scan protocol. Exemplarily, assuming that the name of the pre-scan protocol is an inversion time scout (TI-Scout) sequence, the magnetic resonance device may determine that the type of the pre-scan protocol is the TI-Scout sequence. Exemplarily, assuming that the name of the pre-scan protocol is a velocity encoding with scout (VENC-Scout) sequence, the magnetic resonance device may determine that the type of the pre-scan protocol is the VENC-Scout sequence. Exemplarily, assuming that the name of the pre-scan protocol is a cardiac cine MRI sequence, the magnetic resonance device may determine that the type of the pre-scan protocol is the cardiac cine MRI sequence.

In 704, a plurality of pre-scan images may be obtained by performing scanning on the target object based on the pre-scan protocol, and the plurality of pre-scan images correspond to different pre-scan protocols. In some embodiments, operation 704 may correspond to the operation of obtaining the plurality of pre-scan images by performing a pre-scan hereinabove (e.g., operation 204, operation 404, operation 504, and operation 604).

In an embodiment of the present disclosure, the magnetic resonance device, when formally scanning the target object, may scan the target object based on the pre-scan protocol, i.e., execute the pre-scan protocol on the target object to obtain the plurality of pre-scan images. Each of the plurality of pre-scan images corresponds to a different protocol parameter. It should be noted that the protocol parameter includes, but is not limited to, an inversion time (TI), a velocity coding (VENC), and a trigger delay time of a coronary phase.

In 706, a feature recognition algorithm corresponding to the pre-scan protocol may be determined based on the type of the pre-scan protocol and a pre-stored correspondence relationship between different types of the pre-scan protocol and different feature recognition algorithms.

In some embodiments, operation 706 may correspond to the target parameter determination model in the preceding section, for example, it may be a specific embodiment of an acquisition method of the target parameter determination model in operation 206, operation 406, operation 506, and operation 606 of the preceding section.

It should be noted that the magnetic resonance device has the pre-stored correspondence relationship between the different types of the pre-scan protocols and the different feature recognition algorithms. In this regard, the pre-scan protocol has multiple types, each of which corresponds to a feature recognition algorithm. That is, there is a one-to-one mapping relationship between the types of the pre-scan protocol and the feature recognition algorithms. The feature recognition algorithm may be a neural network-based algorithm.

The feature recognition algorithm includes, but is not limited to a first recognition algorithm for recognizing a myocardial feature of a TI-Scout image, a second recognition algorithm for recognizing curling artifacts of a VENC-Scout image, and a third recognition algorithm for recognizing a coronary motion feature of a cardiac cine MRI sequence.

Exemplarily, taking the first recognition algorithm as an image detection model as an example, the image detection model may be obtained by training an initial neural network model based on a preset sample set. In this case, each sample data in the preset sample set includes a sample TI-Scout image and a TI corresponding to myocardial inversion recovery zero-crossing. In training the initial neural network model, the sample TI-Scout image in each sample is taken as an input of the neural network model, and the TI corresponding to myocardial inversion recovery zero-crossing in each sample is taken as an output of the neural network model. Through the training, the neural network model may learn all the possible correspondence relationships between images and the TIs corresponding to myocardial inversion recovery zero-crossing, and designate a trained neural network model as the image detection model, i.e., obtain the first recognition algorithm.

In some embodiments of the present disclosure, the magnetic resonance device may set a feature recognition algorithm corresponding to the pre-scan protocol of the TI-Scout sequence type as the first recognition algorithm, the magnetic resonance device may set a feature recognition algorithm corresponding to the pre-scan protocol of the VENC-Scout sequence type as the second recognition algorithm, and the magnetic resonance device may set a feature recognition algorithm corresponding to the pre-scan protocol of the cardiac cine MRI sequence type as the third recognition algorithm.

Based on this, after determining the type of the pre-scan protocol, the magnetic resonance device determines, based on the type of the pre-scan protocol and the correspondence relationship between the different types of the pre-scan protocol and the pre-stored different feature recognition algorithms of the magnetic resonance device, the feature recognition algorithm corresponding to the pre-scan protocol.

In 708, a target parameter of a target protocol may be determined by performing feature recognition on the plurality of pre-scan images based on the feature recognition algorithm. In some embodiments, operation 708 may correspond to determining the target parameter in the preceding section, for example, obtaining the first target parameter in operation 206, obtaining the second target parameter in operation 406, obtaining the third target parameter in operation 506, and obtaining the fourth target parameter in operation 606.

In some embodiments of the present disclosure, after the magnetic resonance device determines the feature recognition algorithm corresponding to the pre-scan protocol, since the plurality of pre-scan images are obtained after performing scanning on the target object based on the pre-scan protocol, the magnetic resonance device may obtain, through the determined feature recognition algorithm, an optimal image that meets a first set condition by performing feature recognition on the plurality of pre-scan images, after which the target parameter of the target protocol is determined from the optimal image that meets the first set condition. The first set condition may be set according to the actual need, and is not limited herein.

In some possible embodiments, the first set condition may be set based on the type of pre-scan protocol. Exemplarily, in combination with operation 702, assuming that the type of the pre-scan protocol is the TI-Scout sequence, the first set condition is that the myocardial inversion recovery is zero-crossing, and the optimal image that meets the first set condition is an image of the myocardial inversion recovery zero-crossing. Assuming that the type of the pre-scan protocol is the VENC-Scout sequence, the first set condition is that there are no curling artifacts, and the optimal image that meets the first set condition is an image without curling artifacts. Assuming that the type of the pre-scan protocol is the cardiac cine MRI sequence, the first set condition is the trigger delay time of the coronary phase, and the optimal image that meets the first set condition is a cine MRI image corresponding to the trigger delay time of the coronary phase.

It should be noted that the target protocol includes, but is not limited to a phase sensitive inversion recovery (PSIR) protocol, a flow quantification (FQ) protocol, and a coronary imaging sequence.

Thus, in some other possible embodiments, if the pre-scan protocol is the TI-Scout sequence, the target protocol may be the PSIR protocol. If the pre-scan protocol is the VENC-Scout sequence, the target protocol may be the FQ protocol. If the pre-scan protocol is the cardiac cine MRI sequence, the target protocol may be the coronary imaging sequence.

Based on this, if the type of the pre-scan protocol is the TI-Scout sequence, the target parameter of the target protocol may specifically refer to a TI corresponding to an image of myocardial inversion recovery zero-crossing. If the type of the pre-scan protocol is the VENC-Scout sequence, the target parameter of the target protocol may specifically refer to VENC without curling artifacts. If the type of the pre-scan protocol is the cardiac cine MRI sequence, the target parameter of the target protocol may specifically refer to the trigger delay time of the coronary phase.

In some possible embodiments, there exists a binding identifier or a linking identifier between at least one protocol parameter of the pre-scan protocol and the target parameter of the target protocol. Thus, in the present embodiment, the magnetic resonance device may determine a target protocol relating to the pre-scan protocol based on the binding identifier or the linking identifier and determine a target parameter of the target protocol based on the at least one protocol parameter. The binding identifier or the linking identifier may be set according to actual needs, without limitation here, exemplarily, the binding identifier or the linking identifier may be a number or a digit.

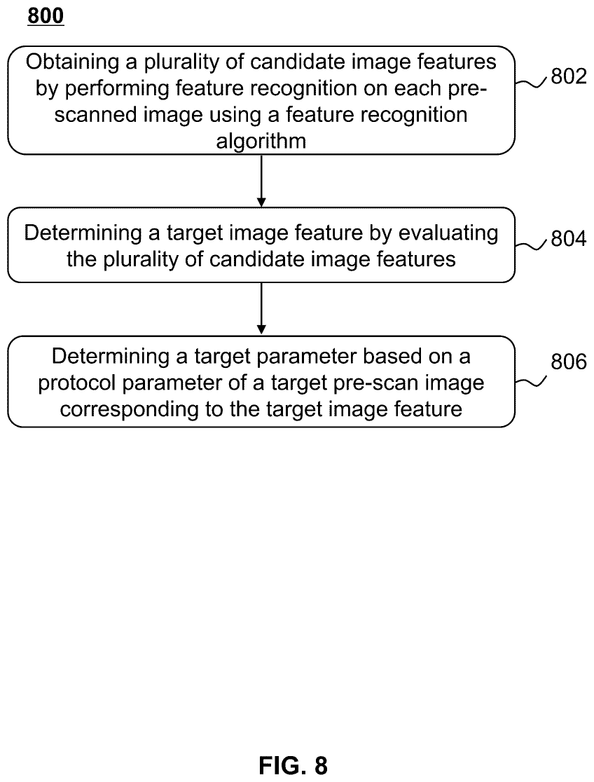

In an embodiment of the present disclosure, in order to improve the accuracy of determining a target protocol parameter, and improve the final imaging quality of the magnetic resonance device, the magnetic resonance device may specifically determine the target protocol parameter through the process 800 as shown in FIG. 8, which may be specifically described in FIG. 8, and the process 800 is a further description of operation 708.

In 710, magnetic resonance scanning may be performed on the target object according to the target parameter.

In some embodiments of the present disclosure, after determining the target parameter of the target protocol, the magnetic resonance device may obtain magnetic resonance data by performing the magnetic resonance scanning on the target object according to the target parameter, and then obtain a magnetic resonance image of the target object by performing magnetic resonance image reconstruction on the magnetic resonance data.

In some embodiments of the present disclosure, the magnetic resonance device may specifically perform operation 710 as below.

If parameter confirmation information entered by a user is detected, the magnetic resonance scanning may be performed on the target object based on the target parameter. The parameter confirmation information may be used to characterize that the user has confirmed the target protocol parameter.

In the present embodiment, after the magnetic resonance device obtains the target parameter of the target protocol, the magnetic resonance device may output a dialog box containing “Whether or not to confirm the target parameter” on a display interface to determine whether the target parameter of the target protocol meets the user's expectation and display corresponding “Yes” control and “No” control.

Based on this, the user may click on any of the above controls on the display interface, i.e., to perform the operation of confirming or not confirming the target parameter, i.e., to input to the magnetic resonance device the information of confirming or not confirming the target parameter.

Exemplarily, if the user clicks on the “Yes” control on the above display screen, the operation of confirming the target parameter is performed, i.e., parameter confirmation information of confirming the target parameter is inputted into the magnetic resonance device. The parameter confirmation information is used to characterize that the user has confirmed the target parameter, i.e., the target parameter is accepted.

Exemplarily, if the user clicks on the “No” control on the display interface, the operation of not confirming the target parameter is performed, i.e., a parameter rejection information of not confirming the target parameter is inputted into the magnetic resonance device. The parameter rejection information is used to characterize the user's rejection of receiving the target parameter.

Based on this, in the present embodiment, the magnetic resonance device, after detecting the parameter confirmation information inputted by the user, indicates that the user has accepted the target parameter, and thus the magnetic resonance device may perform the magnetic resonance scanning on the target object based on the target parameter.

As can be seen above, an embodiment of the present disclosure provides a method for determining the protocol parameter. The method includes obtaining the pre-scan protocol and determining the type of the pre-scan protocol; executing the pre-scan protocol on the target object and obtaining the plurality of pre-scan images, each of the plurality of pre-scan images corresponding to different protocol parameters; flexibly determining the feature recognition algorithm corresponding to the pre-scan protocol based on the type of the pre-scan protocol and the pre-stored correspondence relationship between the different types of the pre-scan protocol and the different feature recognition algorithms; performing image feature recognition on the plurality of pre-scan images based on the feature recognition algorithm to determine the target parameter of the target protocol; and performing the magnetic resonance scanning on the target object based on the target parameter. The method provided in the present disclosure does not require human intervention, and the feature recognition algorithm corresponding to the pre-scan protocol may be flexibly determined in combination with the type of the pre-scan protocol, which improves the accuracy of the determination of the protocol parameter.

FIG. 8 is a flowchart illustrating an exemplary process for determining a target protocol parameter according to some embodiments of the present disclosure. In some embodiments, process 800 may be performed by a system for determining a protocol parameter of cardiac magnetic resonance imaging or a processing device (e.g., the processing device 120). For example, a system for determining a protocol parameter of cardiac magnetic resonance imaging may include at least one storage device including a set of instructions and at least one processor configured to be in communication with the at least one storage device. When the instructions are executed, the at least one processor is configured to instruct the system to perform operations shown in FIG. 8. In some embodiments, the process 800 may include one or more of the operations described below.

In 802, a plurality of candidate image features may be obtained by performing feature recognition on each pre-scanned image using a feature recognition algorithm. In this embodiment, a magnetic resonance device may obtain the plurality of candidate image features by performing the feature recognition on each pre-scanned image using the feature recognition algorithm. The plurality of candidate image features may include, but are not limited to, myocardial features, the presence or absence of curling artifacts and coronary motion features, etc.

It is to be noted that in combination with operation 708, if the pre-scan protocol is a TI-Scout sequence, the candidate image feature is the myocardial feature. If the pre-scan protocol is a VENC-Scout sequence, the candidate image feature is the presence or absence of curling artifacts. If the pre-scan protocol is a cardiac cine MRI sequence, the candidate image feature is a coronary motion feature.

In 804, a target image feature may be determined by evaluating the plurality of candidate image features.

In this embodiment, after obtaining the plurality of candidate image features, the magnetic resonance device may evaluate the plurality of candidate image features based on a first set condition to obtain an optimal candidate image feature among the plurality of candidate image features and designate the optimal candidate image feature as the target image feature. The first set condition may be set according to actual needs and is not limited herein. It should be noted that different types of pre-scan protocols have different first set conditions for evaluating a plurality of corresponding candidate image features.

Combined with operation 802, if the candidate image feature is a myocardial feature, the optimal candidate image feature may be a myocardial zero point. If the candidate image feature is the presence or absence of curling artifacts, the optimal candidate image feature may be the absence of curling artifacts. If the candidate image feature is the coronary motion feature, the optimal candidate image feature may be the trigger delay time of the coronary phase.

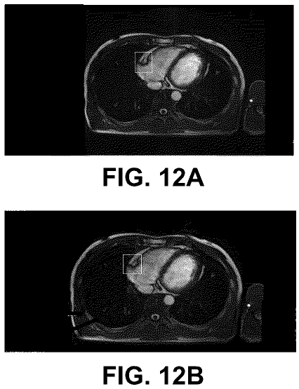

Exemplarily, taking the pre-scan protocol as a cardiac cine MRI sequence an example, referring to FIGS. 12A-12B. FIGS. 12A-12B are schematic diagrams illustrating an exemplary target image feature according to some embodiments of the present disclosure. FIG. 12A represents an image corresponding to a trigger time of the coronary phase, and FIG. 12B represents an image corresponding to a delay time of the coronary phase.

In 806, a target parameter may be determined based on a protocol parameter of a target pre-scan image corresponding to the target image feature. In this embodiment, the magnetic resonance device may designate the protocol parameter of the pre-scan image corresponding to the target image feature, as the target parameter.

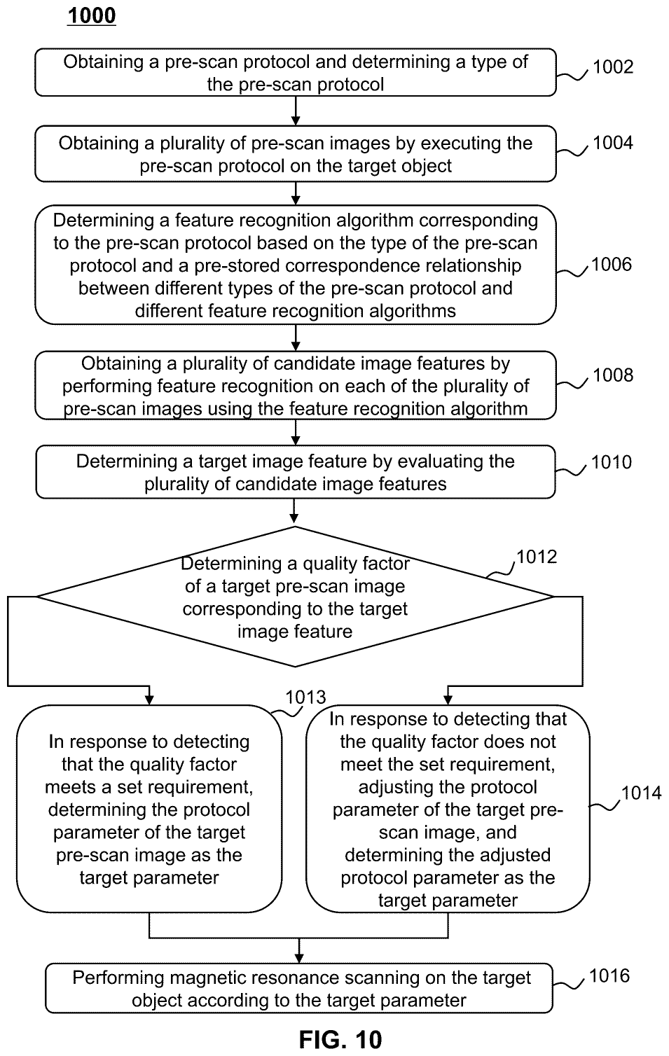

In one embodiment of the present disclosure, in order to ensure the quality of the target parameter of the target protocol to ensure the imaging quality of the subsequent magnetic resonance scanning, the magnetic resonance device may specifically determine the target parameter of the target protocol through a process 1000 as shown in FIG. 10.

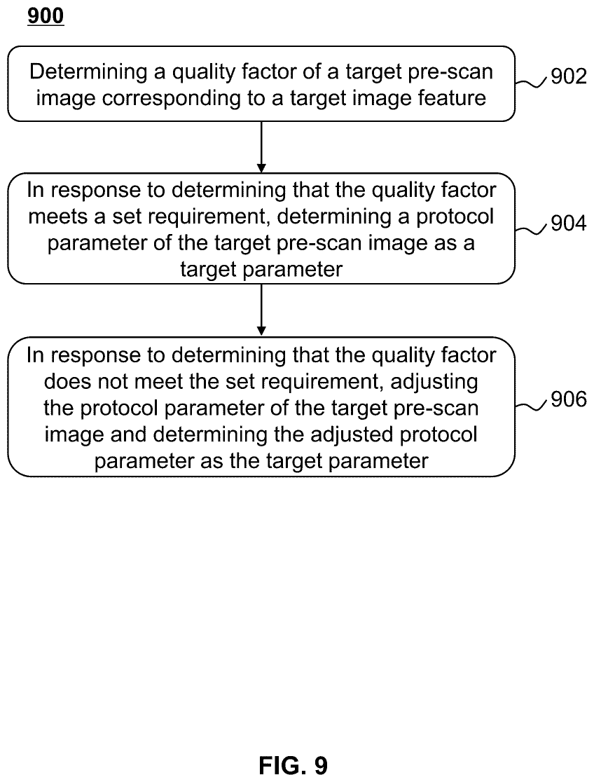

FIG. 9 is a flowchart illustrating an exemplary process for determining a target parameter of a target protocol according to some embodiments of the present disclosure. In some embodiments, the process 900 may be performed by a system for determining a protocol parameter of cardiac magnetic resonance imaging or a processing device (e.g., a processing device 90). For example, a system for determining a protocol parameter of cardiac magnetic resonance imaging may include at least one storage device including a set of instructions and at least one processor configured to be in communication with the at least one storage device. When the instructions are executed, the at least one processor is configured to instruct the system to perform operations shown in FIG. 9. In some embodiments, the process 900 is a further illustration of determining the target parameter of the target protocol, which specifically may include the following operations.

In 902, a quality factor of a target pre-scan image corresponding to a target image feature may be determined.

In practical application, due to various unfavorable factors such as the motion of a target object, cardiac trigger, or inaccurate positioning, the pre-scan image obtained after execution of a pre-scan protocol on the target object may have artifacts, misalignment, poor contrast, etc. At this time, the use of a feature recognition algorithm for feature recognition may have errors, resulting in poor convergence of the algorithm, leading to a low quality factor of the target pre-scan image corresponding to the finally determined target feature image. Therefore, in some embodiments, in order to ensure the quality of the target parameter of the target protocol, the magnetic resonance device may need to determine the quality factor of the target pre-scan image corresponding to the target image feature.

In some embodiments of the present disclosure, the magnetic resonance device may specifically determine the quality factor of the target pre-scan image according to the following operations, as described in more detail below.

Behavior information of the target object may be obtained. If a difference degree between the target pre-scan image and a set image is greater than a second threshold, or if the behavior information does not comply with a second set condition, the quality factor may be determined to be less than a first threshold. If the difference degree between the target pre-scanned image and the set image is less than or equal to the second threshold and the behavior information meets the second set condition, the quality factor may be determined to be greater than or equal to the first threshold.

In this embodiment, the behavior information may be used to characterize whether or not the target object is moving. After the magnetic resonance device obtains the behavior information of the target object, the quality factor of the target pre-scan image may be determined by combining the target pre-scan image and the behavior information.

Specifically, the magnetic resonance device may compare the target pre-scan image with the set image, determine the difference degree between the target pre-scan image and the set image, and compare the difference degree with the second threshold. At the same time, whether or not the behavior information meets the second set condition is detected. The second threshold value may be determined according to actual needs, and is not limited herein.

The set image refers to a standard image that meets the first set condition, and the standard image may be free of artifacts, misalignments, and have high contrast. The high contrast may mean that the contrast of the image meets a benchmark value for determining the contrast according to the actual need, and there is no limitation in the present embodiment relative to the setting of the benchmark value.

In some embodiments, the second set condition may be that the target object is not in motion, or that the target object is at rest.

Based on this, in the present embodiment, the magnetic resonance device, upon detecting that the difference degree between the target pre-scan image and the set image is greater than the second threshold or that the behavior information does not conform to the second set condition, which indicates that the quality factor of the target pre-scan image corresponding to the target image is relatively low, and thus the magnetic resonance device may determine that the quality factor is less than the first threshold. The first threshold may be determined according to actual needs, and is not limited herein.

The magnetic resonance device, upon detecting that the difference degree between the target pre-scan image and the set image is less than or equal to the second threshold and that the behavior information is in accordance with the second set condition, which indicates that the target pre-scan image corresponding to the target image has a relatively high quality factor, and thus the magnetic resonance device may determine that the quality factor is greater than or equal to the first threshold.

In one embodiment of the present disclosure, the magnetic resonance device, when determining that the quality factor is greater than or equal to the first threshold, may determine that the quality factor satisfies a set requirement. Therefore, the magnetic resonance device may perform operation 904.

In another embodiment of the present disclosure, the magnetic resonance device, when determining that the quality factor is less than the first threshold, may determine that the quality factor does not satisfy the set requirement. Therefore, the magnetic resonance device may perform operation 906.

In 904, in response to determining that the quality factor meets the set requirement, the protocol parameter of the target pre-scan image is determined as the target parameter.

In the present embodiment, the magnetic resonance device, when determining that the quality factor of the target pre-scan image meets the set requirement, which indicates that the target image feature corresponds to the target pre-scanning image has a relatively high quality factor. Therefore, the magnetic resonance device may determine the target protocol parameter of the target pre-scan image is determined as the target parameter of the target protocol.

In 906, in response to determining that the quality factor does not meet the set requirement, the protocol parameter of the target pre-scan image may be adjusted, and the adjusted protocol parameter may be determined as the target parameter.

In this embodiment, when the magnetic resonance device determines that the quality factor of the target pre-scan image does not meet the set requirement, which indicates that the target image feature corresponds to the target pre-scan image has a relatively low quality factor. Therefore, the protocol parameter may be adjusted, and the adjusted protocol parameter may be determined as the target parameter of the target protocol.

In one embodiment of the present disclosure, in order to improve a success rate of adjusting the protocol parameter of the target pre-scan image, the magnetic resonance device may specifically adjust the protocol parameter of the target pre-scan image to obtain the target parameter by the following operations, as described in more detail below.

In response to determining that the quality factor is less than the first threshold, the process may return to the operation of performing the pre-scan protocol on the target object to obtain a plurality of pre-scan images, the operation of utilizing the feature recognition algorithm to perform the feature recognition on each of the plurality of the pre-scan images to obtain a plurality of candidate image features, and the operation of evaluating the plurality of candidate image features to determine the target image feature until the quality factor is greater than or equal to the first threshold, or the number of return executions is greater than or equal to a third threshold.

In the present embodiment, when the magnetic resonance device detects that the quality factor of the target pre-scan images is less than the first threshold, which indicates that the quality factor of the target pre-scan images is relatively low, that is, the magnetic resonance device, when executing the pre-scan protocol on the target object, may have patient motion, cardiac trigger, or inaccurate positioning and various other unfavorable factors. Therefore, the magnetic resonance device may re-execute the pre-scan protocol on the target object to obtain a plurality of latest pre-scan images, and then use the feature recognition parameter again to perform the feature recognition on each of the plurality of latest pre-scan images to obtain a plurality of latest candidate image features. In addition, the magnetic resonance device may evaluate the plurality of latest candidate image features to determine a latest target image feature, and determine a quality factor of a target pre-scan image corresponding to the latest target image feature until the quality factor of the target pre-scan image is greater than or equal to the first threshold, or the number of return executions of the magnetic resonance device is greater than or equal to the third threshold. The third threshold may be determined according to actual needs and is not limited herein. Exemplarily, the third threshold may be determined to be 5 times.

In another embodiment of the present disclosure, in order to improve the efficiency of the magnetic resonance device, the magnetic resonance device may specifically adjust the protocol parameter of the target pre-scan image to obtain the target parameter by the following operations, as described in more detail below.

In response to determining that the quality factor is less than the first threshold, parameter adjustment information inputted by the user may be received and the protocol parameter of the target pre-scan image may be adjusted according to the parameter adjustment information, to obtain the target parameter.

In this embodiment, it may indicate that the quality factor of the target pre-scan image is relatively low when the magnetic resonance device detecting that the quality factor of the target pre-scan image is less than the first threshold, i.e., the magnetic resonance device, when executing the pre-scan protocol on the target object, may have various unfavorable factors such as patient motion, cardiac trigger, or inaccurate positioning, etc. Therefore, to improve work efficiency, the magnetic resonance device may directly receive the parameter adjustment information inputted by the user and adjust the protocol parameter of the target pre-scan image according to the parameter adjustment information to obtain the target parameter.

Referring to FIG. 10, FIG. 10 is a flowchart illustrating an exemplary process for determining a protocol parameter according to some embodiments of the present disclosure. In some embodiments, process 1000 may be performed by a system for determining a protocol parameter of cardiac magnetic resonance imaging or a processing device (e.g., the processing device 120). For example, the system for determining the protocol parameter may include at least one storage device including a set of instructions and at least one processor configured to be in communication with the at least one storage device. When the instructions are executed, the at least one processor is configured to instruct the system to perform operations shown in FIG. 10. The process 1000 is a complete embodiment of performing magnetic resonance scanning according to some embodiments of the present disclosure, e.g., embodiments shown in FIGS. 7-10.

As shown in FIG. 10, the magnetic resonance device may perform the following operations after detecting a protocol parameter determination instruction input by a user.

In 1002, a pre-scan protocol may be obtained, and a type of the pre-scan protocol may be determined.

In 1004, a plurality of pre-scan images may be obtained by executing the pre-scan protocol on the target object. Each of the plurality of pre-scan images may correspond to a different protocol parameter.

In 1006, a feature recognition algorithm corresponding to the pre-scan protocol may be determined based on the type of the pre-scan protocol and a pre-stored correspondence relationship between different types of the pre-scan protocol and different feature recognition algorithms.

In 1008, a plurality of candidate image features may be obtained by performing feature recognition on each of the plurality of pre-scan image using the feature recognition algorithm.

In 1010, a target image feature may be determined by evaluating the plurality of candidate image features.

In 1012, a quality factor of a target pre-scan image corresponding to the target image feature may be determined.

On the one hand, the magnetic resonance device, when detecting that the quality factor meets a set requirement, may perform operation 1013 to determine the protocol parameter of the target pre-scan image as the target parameter. On the other hand, the magnetic resonance device, when detecting that the quality factor does not satisfy the set requirement, may perform operation 1014 to adjust the protocol parameter of the target pre-scan image, and determine the adjusted protocol parameter as the target parameter.

In 1016, magnetic resonance scanning may be performed on the target object according to the target parameter.

It should be understood that the serial numbers of the operations in the above-described embodiments does not imply the order of execution, and the order of execution of the processes should be determined by their functions and inherent logic without constituting any limitation of the process of implementing the embodiments of the present disclosure.

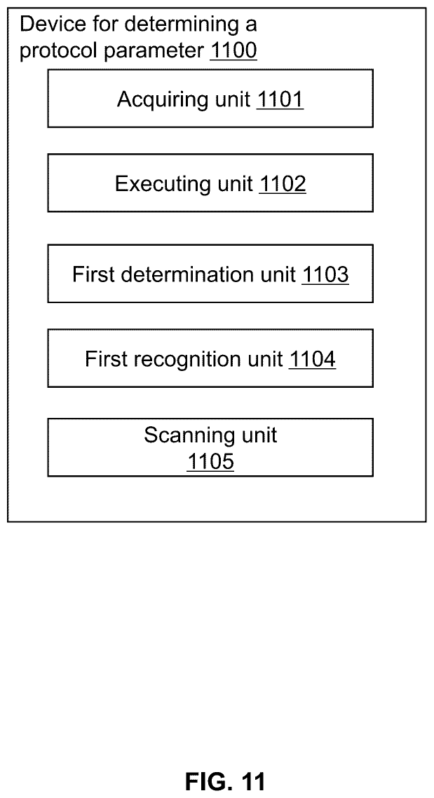

Based on a method for determining a protocol parameter described in the above embodiment, FIG. 11 is a schematic diagram illustrating a device for determining a protocol parameter according to some embodiments of the present disclosure. For the sake of illustration, only a portion related to the embodiments of the present disclosure is shown portions that are relevant to the embodiments of the present disclosure. Referring to FIG. 11, a device 1100 for determining the protocol parameter may include an acquiring unit 1101, an executing unit 1102, a first determination unit 1103, a first recognition unit 1104, and a scanning unit 1105.

The acquisition unit 1101 may be configured to acquire a pre-scan protocol and determine a type of the pre-scan protocol.

An executing unit 1102 may be configured to execute the pre-scan protocol on the target object to obtain a plurality of pre-scan images, each of the plurality of pre-scan images corresponding to a different protocol parameter.

The first determination unit 1103 may be configured to determine the feature recognition algorithm corresponding to the pre-scan protocol based on the type of the pre-scan protocol and a pre-stored correspondence relationship between different types of the pre-scan protocol and different feature recognition algorithms.

The first recognition unit 1104 may be configured to characterize the plurality of pre-scan images according to the feature recognition algorithm to determine the target parameter of the target protocol.

The scanning unit 1105 may be configured to perform magnetic resonance scanning on a target object based on the target parameter.

In an embodiment of the present disclosure, the first recognition unit 1104 may specifically include a second recognition unit, an evaluation unit, and a second determination unit. The second recognition unit may be configured to obtain a plurality of candidate image features by performing feature recognition on each of the plurality of pre-scan images using the feature recognition algorithm. The evaluation unit may be configured to determine a target image feature by evaluating the plurality of candidate image features. The second determination unit may be configured to determine the target parameter based on the protocol parameter of a target pre-scan image corresponding to the target image feature. In one embodiment of the present disclosure, the second determination unit may specifically include a third determination unit, a fourth determination unit and a fifth determination unit. The third determination unit is configured to determine the quality factor of the target pre-scan image corresponding to the target image feature. The fourth determination unit may be configured to determine the protocol parameter of the target pre-scan image as the target parameter in response to the quality factor meeting a set requirement. The fifth determination unit may be configured to adjust the protocol parameter of the target pre-scan image in response to the quality factor not meeting the set requirement, and to determine the adjusted protocol parameter as the target parameter.

In one embodiment of the present disclosure, the pre-scan protocol may be a TI-Scout sequence, each of the plurality of candidate image features may be a myocardial feature, and the target image feature may be a myocardial zero point. Correspondingly, the target protocol may be a phase sensitive inversion recovery (PSIR) protocol and the target parameter is a TI.

In one embodiment of the present disclosure, the pre-scan protocol may be a VENC-Scout sequence, and each of the plurality of candidate image features may be the presence or absence of curling artifacts. Correspondingly, the target protocol may be a FQ protocol, and the target parameter may be a VENC.

In one embodiment of the present disclosure, the pre-scan protocol may be a cardiac cine MRI sequence, each of the plurality of candidate image features may be a coronary motion feature. The target protocol may refer to a coronary imaging sequence, and the target parameter may be a trigger delay time of a coronary phase.

In one embodiment of the present disclosure, there exists a binding identifier or a linking identifier between at least one protocol parameter of the pre-scan protocol and the target parameter of the target protocol.

In one embodiment of the present disclosure, the feature recognition algorithm may be a neural network-based algorithm.

It is to be noted that the contents of the information interaction, execution process, etc. between the above-mentioned devices or units, since they are based on the same idea as the method embodiments of the present disclosure, and specific functions and technical effects brought about may be seen in detail in the section of method embodiments, and will not be repeated here.

In some embodiments of the present disclosure, the magnetic resonance device may further include a display for displaying the target parameter as determined by any one of the method embodiments described above.

It should be noted that the target parameter is also set with a binding identifier or linking identifier, which is used to characterize that the target parameter is in a reuse relationship with at least one of the parameters of the pre-scan protocol.

It should be noted that the foregoing descriptions with respect to the respective processes are for the purpose of exemplification and illustration only and do not limit the scope of application of the present disclosure. For a person skilled in the art, various corrections and changes can be made to the respective processes under the guidance of the present disclosure. However, these corrections and changes remain within the scope of the present disclosure. For example, adding storage operations between processes, etc.

It should be understood that the system shown in FIG. 1 and its modules can be implemented utilizing a variety of approaches. For example, in some embodiments. . . .

It is to be noted that the above description of the system for determining the protocol parameter and its modules is for descriptive convenience only, and does not limit the present disclosure to the scope of the cited embodiments. It is to be understood that for a person skilled in the art, after understanding the principle of the system, it may be possible to arbitrarily combine the individual modules or form a sub-system to be connected to the other modules without departing from the principle. In some embodiments, the modules described above may be different modules in a single system, or a single module may implement the functions of two or more of the modules described above. For example, the individual modules may share a common storage module, and the individual modules may each have a respective storage module. Morphisms such as these are within the scope of protection of the present disclosure.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A system for determining a protocol parameter of cardiac magnetic resonance imaging, comprising:
at least one storage device storing a set of instructions; and
at least one processor configured to communicate with the at least one storage device, and when executing the set of instructions, the at least one processor is configured to direct the system to perform operations including:
in response to detecting that a user operation satisfies a first condition, obtaining an inversion time scout (TI-Scout) sequence for cardiac scanning;
obtaining a plurality of first scan images by performing a first scan on a target object based on the TI-Scout sequence protocol; and
generating a first target parameter for a phase sensitive inversion recovery (PSIR) protocol using a target parameter determination model based on the plurality of first scan images, the first target parameter including an inversion time (TI) corresponding to an image of myocardial inversion recovery zero-crossing, and the target parameter determination model being a machine learning model.

2. The system of claim 1, wherein the target parameter determination model includes an image pre-processing layer and a first image evaluation layer, and the generating a first target parameter for a PSIR protocol using a target parameter determination model based on the plurality of first scan images includes:
generating a candidate image sequence based on the plurality of first scan images through the image pre-processing layer; and
determining the first target parameter based on the candidate image sequence and a preset evaluation metric through the first image evaluation layer.

3. The system of claim 1, wherein the target parameter determination model includes a feature extraction layer and a second image evaluation layer, and the generating a first target parameter for a PSIR protocol using a target parameter determination model based on the plurality of first scan images includes:
determining a first preset feature based on the plurality of first scan images;
determining a candidate image feature sequence based on the first preset feature and the plurality of first scan images through the feature extraction layer; and
determining the first target parameter based on the candidate image feature sequence through the second image evaluation layer.

4. The system of claim 3, wherein an input of the target parameter determination model further includes historically and manually selected protocol parameters.

5. The system of claim 1, wherein the first condition includes:
detecting a user's operation of selecting a first binding group, the first binding group reflecting a binding relationship between at least one parameter of the TI-Scout sequence protocol and the first target parameter.

6. The system of claim 1, wherein the operations further include:
in response to detecting that the user's operation satisfies a second condition, obtaining a velocity encoding with scout (VENC-Scout) sequence protocol for cardiac scanning;
obtaining a plurality of second scan images by performing a second scan on the target object based on the VENC-Scout sequence protocol; and
generating a second target parameter for a flow quantification (FQ) protocol using the target parameter determination model based on the plurality of second scan images, the second target parameter including VENC without curling artifacts.

7. The system of claim 1, wherein the operations further include:
in response to detecting that the user's operation satisfies a third condition, obtaining a cardiac cine MRI sequence protocol for cardiac scanning;
obtaining a plurality of third scan images by performing a third scan on the target object based on the cardiac cine MRI sequence protocol; and
generating a third target parameter for a coronary imaging sequence protocol using the target parameter determination model based on the plurality of third scan images, the third target parameter including a trigger delay time of a coronary phase.

8. The system of claim 1, wherein the operations further include:
in response to detecting that the user's operation satisfies a fourth condition, obtaining a bolus tracking sequence protocol for vascular scanning;
obtaining a plurality of fourth scan images by performing a fourth scan on the target object based on the bolus tracking sequence protocol; and
generating a fourth target parameter for a vascular sequence protocol using the target parameter determination model based on the plurality of fourth scan images, the fourth target parameter including an instruction to automatically send a confirmation for subsequent scanning when an intensity of a vascular target region in the plurality of fourth scan images reaches an expected level.

9. The system of claim 1, further comprising a display for displaying the first target parameter marked with a binding identifier or a linking identifier, the binding identifier or the linking identifier being configured to indicate the first target parameter is shared with at least another sequence protocol.

10. A method for determining a protocol parameter, comprising:
in response to detecting that a user operation satisfies a first condition, obtaining an inversion time scout (TI-Scout) sequence for cardiac scanning;
obtaining a plurality of first scan images by performing a first scan on a target object based on the TI-Scout sequence protocol; and
generating a first target parameter for a phase sensitive inversion recovery (PSIR) protocol using a target parameter determination model based on the plurality of first scan images, the first target parameter including an inversion time (TI) corresponding to an image of myocardial inversion recovery zero-crossing, and the target parameter determination model being a machine learning model.

11. The method of claim 10, wherein the target parameter determination model includes an image pre-processing layer and a first image evaluation layer, and the generating a first target parameter for a PSIR protocol using a target parameter determination model based on the plurality of first scan images includes:
generating a candidate image sequence based on the plurality of first scan images through the image pre-processing layer; and
determining the first target parameter based on the candidate image sequence and a preset evaluation metric through the first image evaluation layer.

12. The method of claim 10, wherein the target parameter determination model includes a feature extraction layer and a second image evaluation layer, and the generating a first target parameter for a PSIR protocol using a target parameter determination model based on the plurality of first scan images includes:
determining a first preset feature based on the plurality of first scan images;
determining a candidate image feature sequence based on the first preset feature and the plurality of first scan images through the feature extraction layer; and
determining the first target parameter based on the candidate image feature sequence through the second image evaluation layer.

13. The method of claim 12, wherein an input of the target parameter determination model further includes historically and manually selected protocol parameters.

14. The method of claim 10, wherein the first condition includes:
detecting a user's operation of selecting a first binding group, the first binding group reflecting a binding relationship between at least one parameter of the TI-Scout sequence protocol and the first target parameter.

15. The method of claim 10, wherein the operations further include:
in response to detecting that the user's operation satisfies a second condition, obtaining a velocity encoding with scout (VENC-Scout) sequence protocol for cardiac scanning;
obtaining a plurality of second scan images by performing a second scan on the target object based on the VENC-Scout sequence protocol; and
generating a second target parameter for a flow quantification (FQ) protocol using the target parameter determination model based on the plurality of second scan images, the second target parameter including VENC without curling artifacts.

16. The method of claim 10, wherein the operations further include:
in response to detecting that the user's operation satisfies a third condition, obtaining a cardiac cine MRI sequence protocol for cardiac scanning;
obtaining a plurality of third scan images by performing a third scan on the target object based on the cardiac cine MRI sequence protocol; and
generating a third target parameter for a coronary imaging sequence protocol using the target parameter determination model based on the plurality of third scan images, the third target parameter including a trigger delay time of a coronary phase.

17. The method of claim 10, wherein the operations further include:
in response to detecting that the user's operation satisfies a fourth condition, obtaining a bolus tracking sequence protocol for vascular scanning;
obtaining a plurality of fourth scan images by performing a fourth scan on the target object based on the bolus tracking sequence protocol; and
generating a fourth target parameter for a vascular sequence protocol using the target parameter determination model based on the plurality of fourth scan images, the fourth target parameter including an instruction to automatically send a confirmation for subsequent scanning when an intensity of a vascular target region in the plurality of fourth scan images reaches an expected level.

18. A method for cardiac scan during magnetic resonance imaging comprising:
obtaining a pre-scan protocol;
obtaining a plurality of pre-scan images by performing a scan on a heart of the object based on the pre-scan protocol, the plurality of pre-scan images corresponding to a series of parameter values of the pre-scan protocol;
determining a target parameter of a target protocol by performing feature recognition on the plurality of pre-scan images using a deep learning-based network; and
performing magnetic resonance scanning on the heart of the object according to the target protocol;
wherein a type of the pre-scan protocol is different from that of the target protocol.

19. The method of claim 18, wherein the determining a target parameter of a target protocol by performing feature recognition on the plurality of pre-scan images using a deep learning-based network includes:
obtaining a plurality of candidate image features by performing feature recognition on each of the plurality of pre-scan images using a feature recognition algorithm;
determining a target image feature by evaluating the plurality of candidate image features; and
determining the target parameter based on a protocol parameter of a target pre-scan image corresponding to the target image feature.

20. The method of claim 19, wherein the determining the target parameter based on a protocol parameter of a target pre-scan image corresponding to the target image feature includes:
determining a quality factor of the target pre-scan image corresponding to the target image feature;
in response to determining that the quality factor meets a set requirement, determining the protocol parameter of the target pre-scan image as the target parameter; and
in response to determining that the quality factor does not meet the set requirement, adjusting the protocol parameter of the target pre-scan image and determining the adjusted protocol parameter as the target parameter.
